(19) United States
(12) Patent Application Publication
(10) Pub. No.: US 2008/0027010 A1
(43) Pub. Date: Jan. 31, 2008

(54) Treatment of refractory cancers using Na+/K+-ATPase inhibitors

(75) Inventors: Mehran Khodadoust, Brookline, MA (US); Ajay Sharma, Sudbury, MA (US); Reimar Bruening, Frermont, CA (US)

Correspondence Address: ROPES & GRAY LLP;PATENT DOCKETING 39/41, ONE INTERNATIONAL PLACE, BOSTON, MA, 02110-2624, US

(73) Assignee: Bionaut Pharmaceuticals, Inc.

(21) Appl. No.: 11/653,076

(22) Filed: Jan. 12, 2007

Related U.S. Application Data
(63) Continuation of application No. 11/441,394, filed on May 24, 2006, now abandoned.
Application No. 11/441,394 is a continuation-in-part of application No. 11/218,332, filed on Sep. 1, 2005.
(60) Provisional application No. 60/606,777, filed on Sep. 2, 2004.

(51) Int. Cl. A61K 31/57; A61K 31/704; A61P 35/00

(52) U.S. Cl. 514/26; 514/171; 514/182

(57) ABSTRACT

The reagent, pharmaceutical formulation, kit, and methods of the invention provides a new approach to treat refractory cancers using Na⁺/K⁺-ATPase inhibitors, such as cardiac glycosides, including bufadienolides or their corresponding aglycones (e.g., proscillaridin, scillaren, and scillarenin, etc.), especially in oral formulations and/or solid dosage forms containing more than 1 mg of active ingredients.

### REFERENCE TO RELATED APPLICATION

This application is a continuation-in-part application of U.S. Ser. No. 11/218,332, filed on Sep. 1, 2005, which claims the benefit of the filing date of U.S. Provisional Application Ser. No. 60/606,777, entitled “TREATMENTS OF REFRACTORY CANCERS USING CARDIAC GLYCOSIDES AND OTHER Na⁺/K⁺-ATPASE INHIBITORS,” and filed on Sep. 2, 2004. The teachings of the referenced applications are incorporated herein by reference.

### BACKGROUND OF THE INVENTION

Clinical drug resistance, either intrinsic or acquired, is a major barrier to overcome before chemotherapy can become curative for most patients presenting with cancer. In many common cancers (for example, non-small cell lung, testicular and ovarian cancers), substantial tumor shrinkage can be expected in more than 50% of cases with conventional chemotherapy. In other cases, response rates are lower; 10-20% of patients with renal cell carcinoma, pancreatic and esophageal cancers respond to treatment. In almost all cases, drug resistance eventually develops shortly and is often fatal. If this could be treated, prevented or overcome, the impact would be substantial.

Such resistance or refractory phenotype may be brought about by a variety of mechanisms. For example, there is (i) p-gylocoprotein mediated multi-drug resistance (MDR); (ii) mutant topoisomerase mediated atypical MDR; (iii) tubulin mutation mediated resistance to taxanes; and (iv) resistance to cisplatin.

In addition, response of certain tumors to conventional chemotherapy and/or radio therapy may also contribute to refractory cancer by promoting cellular stress responses such as induction of the hypoxic response as visualized via HIF-1 expression. HIF-1 is a transcription factor and is critical to cancer survival in hypoxic conditions. HIF-1 is composed of the O₂— and growth factor-regulated subunit HIF-1α, and the constitutively expressed HIF-1β subunit (arylhydrocarbon receptor nuclear translocator, ARNT), both of which belong to the basic helix-loop-helix (bHLH)-PAS (PER, ARNT, SIM) protein family. So far in the human genome 3 isoforms of the subunit of the transcription factor HIF have been identified: HIF-1, HIF-2 (also referred to as EPAS-1, MOP2, HLF, and HRF), and HIF-3 (of which HIF-32 also referred to as IPAS, inhibitory PAS domain).

Under normoxic conditions, HIF-1α a is targeted to ubiquitinylation by pVHL and is rapidly degraded by the proteasome. This is triggered through posttranslational HIF-hydroxylation on specific proline residues (proline 402 and 564 in human HIF-1α protein) within the oxygen dependent degradation domain (ODDD), by specific HIF-prolyl hydroxylases (HPH1-3 also referred to as PHD1-3) in the presence of iron, oxygen, and 2-oxoglutarate. The hydroxylated protein is then recognized by pVHL, which functions as an E3 ubiquitin ligase. The interaction between HIF-1α and pVHL is further accelerated by acetylation of lysine residue 532 through an N-acetyltransferase (ARD1). Concurrently, hydroxylation of the asparagine residue 803 within the C-TAD also occurs by an asparaginyl hydroxylase (also referred to as FIH-1), which by its turn does not allow the coactivator p300/CBP to bind to HIF-1α subunit. In hypoxia HIF-1α remains not hydroxylated and stays away from interaction with pVHL and CBP/p300 (FIG. 6). Following hypoxic stabilization HIF-1α translocates to the nucleus where it hetero-dimerizes with HIF-1β. The resulting activated HIF-1 drives the transcription of over 60 genes important for adaptation and survival under hypoxia including glycolytic enzymes, glucose transporters Glut-1 and Glut-3, endothelin-1 (ET-1), VEGF (vascular endothelial growth factor), tyrosine hydroxylase, transferrin, and erythropoietin (Brahimi-Horn et al., 2001 Trends Cell Biol 11 (11): S32-S36; Beasley et al., 2002 Cancer Res 62(9): 2493-2497; Fukuda et al., 2002 J Biol Chem 277(41): 38205-38211; Maxwell and Ratcliffe, 2002 Semin Cell Dev Biol 13(1): 29-37).

Regardless of the mechanism, refractory cancer is a serious problem because it signals the failure of conventional cancer therapy. It is an object of the present invention to provide a novel and more effective approach to treat cancers refractory to conventional chemotherapy.

### SUMMARY OF THE INVENTION

The inventors have discovered that certain anti-tumor agents, in addition to their cancer-killing effects, in fact also promote stress responses in tumor cells. Such stress response protects cells from programmed cell death and promotes tumor growth, by promoting cell survival through induction of growth factors and pro-angiogenesis factors, and by activating anaerobic metabolism, which have a direct negative consequence on clinical and prognostic parameters, and create a therapeutic challenge, including refractory cancer.

The hypoxic response includes induction of HIF-1-dependent transcription, which exerts complex effect on tumor growth, and involves the activation of several adaptive pathways.

Through the use of cellular assays that report a cells response to stress, the inventors have discovered for the first time that Na⁺/K⁺-ATPase inhibitors (such as the cardenolide cardiac glycoside Ouabain, and, to an even larger degree, the bufadienolide cardiac glycoside BNC-4 (i.e., Proscillaridin), and their respective aglycones) induce a signal that prevents cancer cells to respond to stresses such as hypoxic stress through transcriptional inhibition of Hypoxia Inducible Factor (HIF-1α) biosynthesis.

The inventors have discovered that the cellular and systemic responses share common endogenous cardiac glycosides, including ouabain and proscillaridin. However, the inventors also found that cardiac glycosides serve different roles in the cellular and systemic responses to hypoxic stress. Specifically, at the system level, cardiac glycosides are produced to mediate the body's response to hypoxic stress, including a role in regulating heart rate and increasing blood pressure associated with chronic hypoxic stress. Thus, endogenous cardiac glycosides' properties as mediators of such systemic response to hypoxia have been explored in the development of cardiovascular medications. Cardiac glycosides used in such medications, such as digoxin, ouabain and proscillaridin, are steroidal compounds chemically identical to endogenous cardiac glycosides.

In contrast, at the cellular level, cardiac glycosides inhibit a cell from making its normal survival response to hypoxic conditions, e.g., VEGF secretion, and theoretically enable the body to conserve limited resources so as to ensure the survival of the major organs. These findings demonstrate the existence of a cellular regulatory pathway that can modulate a cell's response to stress, the modulation of which cellular regulatory pathway may provide novel, effective treatment methods, such as the treatment of cancers. These findings also demonstrate a novel role for the systemic mediator of the body's response to hypoxic stress (e.g., the cardiac glycosides) in modulating normal cellular responses to hypoxia.

While not wishing to be bound by any particular theory, these Na⁺/K⁺-ATPase inhibitors at the cellular level bind to the sodium-potassium channel (Na⁺/K⁺-ATPase), and induces a signal that results in anti-proliferative events in cancer cells. This binding and signaling event proceeds independently from the pump-inhibition effect of these Na⁺/K⁺-ATPase inhibitors, and thus presents a novel mechanism for cancer treatment. Therefore, this discovery forms one basis for using cardiac glycosides (such as Proscillaridin, and their aglycones) in anti-cancer therapy. The anti-cancer therapy of the instant invention is useful in treating refractory cancers, especially those HIF-1α-associated refractory cancers.

Thus a salient feature of the present invention is the discovery that Na⁺/K⁺-ATPase inhibitors, such as cardiac glycosides, can be used to effectively treat at least certain cancers refractory to conventional chemo- and/or radio-therapy.

Thus one aspect of the invention provides a method of inhibiting the growth or spread of a refractory cancer in an individual, comprising administering to the individual a Na⁺/K⁺-ATPase inhibitor in an oral dosage form over a treatment period.

In a related aspect, the invention provides a use of a Na⁺/K⁺-ATPase inhibitor in the manufacture of a medicament in oral dosage form, for treating/inhibiting the growth or spread of a refractory cancer in an individual over a treatment period.

Another aspect of the invention provides a method for promoting treatment of an individual suffering from a refractory cancer, comprising packaging, labeling and/or marketing a Na⁺/K⁺-ATPase inhibitor in an oral dosage form to be used as part of a treatment for inhibiting the growth or spread of the refractory cancer over a treatment period.

In a related aspect, the invention provides a use of a Na⁺/K⁺-ATPase inhibitor in the packaging, labeling and/or marketing of an oral dosage form medicament, for treating/inhibiting the growth or spread of a refractory cancer in an individual over a treatment period.

Another aspect of the invention provides a method of treating multidrug resistance of refractory tumor cells in a refractory cancer patient in need of such treatment, said method comprising administering, concurrently or sequentially, an effective amount of a Na⁺/K⁺-ATPase inhibitor in an oral dosage form and an anti-neoplastic agent to said patient.

In a related aspect, the invention provides a use of a Na⁺/K⁺-ATPase inhibitor in the manufacture of a medicament in oral dosage form, for treating multidrug resistance of refractory tumor cells in a refractory cancer patient in need of such treatment, the Na⁺/K⁺-ATPase inhibitor being administered, concurrently or sequentially, with an anti-neoplastic agent to the patient.

Another aspect of the invention provides a packaged pharmaceutical comprising a Na⁺/K⁺-ATPase inhibitor formulated as an oral dosage form in a pharmaceutically acceptable excipient and suitable for use in humans, and optionally a label or instructions for administering the Na⁺/K⁺-ATPase inhibitor as part of a treatment for inhibiting the growth or spread of a refractory cancer.

Another aspect of the invention provides a pharmaceutical composition comprising a bufadienolide Na⁺/K⁺-ATPase inhibitor or aglycone thereof, formulated in a pharmaceutically acceptable excipient and suitable for use in humans, the bufadienolide or aglycone thereof is a solid oral dosage form of at least about 1.5 mg, about 2.0 mg, about 2.25 mg, about 2.5 mg, about 3.0 mg, about 4.0 mg, about 5.0 mg, about 7.5 mg, about 10 mg, or about 15 mg.

For any of the aspects of the invention described herein, the following embodiments, each independent of one another as appropriate, and is able to combine with any of the other embodiment when appropriate, are contemplated below.

For any of the different aspects of the invention, the cancer may be refractory to radiation therapy, or refractory to anti-cancer chemotherapy. In one embodiment, the cancer may be refractory to anti-cancer chemotherapy, but not refractory to radiation therapy.

The refractory cancer may be a solid tumor, such as a tumor in the pancreas, lung, kidney, ovarian, breast, prostate, stomach, colon, bladder, prostate, brain, skin, testicles, cervix, uterine, bone, or liver. The solid tumor may be a pancreatic tumor refractory to treatment by one or more of: fluorouracil, carmustine (BCNU), temozolomide (TMZ), streptozotocin, and gemcitabine. The solid tumor may be a lung tumor refractory to etoposide or platinum-based therapy. For example, the lung tumor may be refractory small cell lung cancer, or refractory non-small cell lung cancer. The refractory cancer may also be a hematological cancer, such as one selected from: acute lymphoblastic leukemia (ALL), acute lymphoblastic B-cell leukemia, acute lymphoblastic T-cell leukemia, acute nonlymphoblastic leukemia (ANLL), acute myeloblastic leukemia (AML), acute promyelocytic leukemia (APL), acute monoblastic leukemia, acute erythro-leukemic leukemia, acute megakaryoblastic leukemia, chronic myelocytic leukemia (CML), chronic lymphocytic leukemia (CLL), multiple myeloma, myelodysplastic syndrome (MDS), or chronic myelo-monocytic leukemia (CMML), wherein MDS may be either refractory anemia with excessive blast (RAEB) or RAEB in transformation to leukemia (RAEB-T).

In certain embodiments, the Na⁺/K⁺-ATPase inhibitor is a cardiac glycoside or an aglycone thereof. For example, the cardiac glycoside may have an IC₅₀ for killing one or more different cancer cell lines of 500 nM or less, and even more preferably 200 nM, 100 nM, 10 nM or even 1 nM or less.

In certain embodiments, the Na⁺/K⁺-ATPase inhibitor has a therapeutic index of at least about 2, preferably at least about 3, 5, 8, 10, 15, 20, 25, 30, 40, or about 50. Therapeutic index refers to the ratio between the minimum toxic serum concentration of a compound, and a therapeutically effective serum concentration sufficient to achieve a pre-determined therapeutic end point. For example, the therapeutic end point may be >50% or 60% inhibition of tumor growth (compared to an appropriate control) in a xenograph nude mice model, or in clinical trial.

In certain embodiments, the treatment period is about 1 month, 3 months, 6 months, 9 months, 1 year, 3 years, 5 years, 10 years, 15 years, 20 years, or the life-time of the individual.

In certain embodiments, the oral dosage form maintains an effective steady state serum concentration of about 10-100 ng/mL, about 15-80 ng/mL, about 20-50 ng/mL, or about 20-30 ng/mL.

In certain embodiments, the steady state serum concentration is reached by administering a total dose of about 5-10 mg/day, and a continuing dose(s) of about 1.5-5 mg/day in a human individual, preferably over the subsequent 1-3 days.

In certain embodiments, the oral dosage form comprises a total daily dose of about 1-7.5 mg, about 1.5-5 mg, or about 3-4.5 mg per human individual.

In certain embodiments, the oral dosage form is a solid oral dosage form.

In certain embodiments, the oral dosage form comprises a daily dose of 2-3 times of 1.5 mg cardiac glycoside or an aglycone thereof.

Unless otherwise indicated, the total daily dose may be administered as a single dose, or in as many doses as the physicians may choose.

In certain embodiments, the total daily dose may be administered as a single dose for, e.g., patient convenience, and/or better patient compliance.

In certain embodiments, the Cₘₐₓ is kept low by administering the total daily dosage over multiple doses (e.g., 2-5 doses, or 3 doses). This may be beneficial for patients who exibits certain side effects such as nausea and vomiting, for patients with weak heart muscles, or who otherwise do not tolerate relatively high doses or Cₘₐₓ well.

In certain embodiments, the oral dosage form comprise a single solid dose of about 1 mg, 1.5 mg, 2 mg, 2.5 mg, 3 mg, 3.5 mg, 4 mg, 4.5 mg, 5 mg, 5.5 mg, 6 mg, 6.5 mg, or about 7 mg of active ingredient.

In certain embodiments, the cardiac glycoside or aglycone thereof is represented by the general formula:

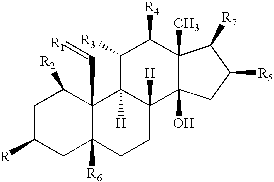

wherein

R represents a glycoside of 1 to 6 sugar residues, or —OH;

R₁ represents H, H; H, OH; or ═O;

R₂, R₃, R₄, R₅, and R₆ each independently represents hydrogen or —OH; and,

R₇ represents

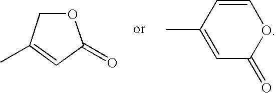

The sugar residues may be selected from: L-rhamnose, D-glucose, D-digitoxose, D-digitalose, D-digginose, D-sarmentose, L-vallarose, or D-fructose. These sugars may be in the β-conformation. The sugar residues may be acetylated, e.g., to effect the lipophilic character and the kinetics of the entire glycoside. The glycoside may be 1-4 or 1-6 sugar residues in length.

In certain embodiments, the cardiac glycoside may comprise a steroid core with either a pyrone substituent at C17 (the “bufadienolides form”), or a butyrolactone substituent at C17 (the “cardenolide” form).

In certain embodiments, the cardiac glycoside is a bufadienolide comprising a steroid core with a pyrone substituent R7 at C17. The cardiac glycoside may have an IC₅₀ for killing one or more different cancer cell lines of about 500 nM, 200 nM, 100 nM, 10 nM or even 1 nM or less.

In certain embodiments, the cardiac glycoside is proscillaridin (e.g., Merck Index registry number 466-06-8) or scillaren (e.g., Merck Index registry number 11003-70-6).

In certain embodiments, the aglycone is scillarenin (e.g., Merck Index registry number 465-22-5).

In certain embodiments, the cardiac glycoside may be selected from: digitoxigenin, digoxin, lanatoside C, Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin, calotoxin, convallatoxin, oleandrigenin, bufalin, periplocymarin, digoxin (CP 4072), strophanthidin oxime, strophanthidin semicarbazone, strophanthidinic acid lactone acetate, ernicyrnarin, sannentoside D, sarverogenin, sarmentoside A, sarmentogenin, or a pharmaceutically acceptable salt, ester, amide, or prodrug thereof.

In certain embodiments, the cardiac glycoside is ouabain or proscillaridin.

Other Na⁺/K⁺-ATPase inhibitors are available in the literature. See, for example, U.S. Pat. No. 5,240,714, which describes a non-digoxin-like Na⁺/K⁺-ATPase inhibitory factor. Recent evidence suggests the existence of several endogenous Na⁺/K⁺-ATPase inhibitors in mammals and animals. For instance, marinobufagenin (3,5-dihydroxy-14,15-epoxy bufodienolide) may be useful in the current combinatorial therapies.

Those skilled in the art can also rely on screening assays to identify compounds that have Na⁺/K⁺-ATPase inhibitory activity. PCT Publications WO00/44931 and WO02/42842, for example, teach high-throughput screening assays for modulators of Na⁺/K⁺-ATPases.

The Na⁺/K⁺-ATPase consists of at least two dissimilar subunits, the large α subunit with all known catalytic functions and the smaller glycosylated β subunit with chaperonic function. In addition there may be a small regulatory, so-called FXYD peptide. Four a peptide isoforms are known and isoform-specific differences in ATP, Na⁺ and K⁺ affinities and in Ca²⁺ sensitivity have been described. Thus changes in Na⁺/K⁺-ATPase isoform distribution in different tissues, as a function of age and development, electrolytes, hormonal conditions etc. may have important physiological implications. Cardiac glycosides like ouabain are specific inhibitors of the Na⁺/K⁺-ATPase. The four a peptide isoforms have similar high ouabain affinities with K_{d} of around 1 nM or less in almost all mammalian species. In certain embodiments, the Na⁺/K⁺-ATPase inhibitor is more selective for complexes expressed in non-cardiac tissue, relative to cardiac tissue.

In certain embodiments, the resistance of the refractory cancer to a therapeutic agent is mediated through tubulin.

In certain embodiments, the resistance of the refractory cancer to a therapeutic agent is mediated through multidrug resistance.

In certain embodiments, the multidrug resistance is caused by increased expression of ATP-binding cassette (ABC) transporters; overexpression of P-gp; or changes in topoisomerase II, protein kinase C or specific glutathione transferase enzymes.

In certain embodiments, the resistance of the refractory cancer to a therapeutic agent is mediated through topoisomerase.

In certain embodiments, the resistance of the refractory cancer to a therapeutic agent is mediated through Mitoxantrone.

In certain embodiments, the subject cardiac glycoside may be conjointly administered with an effective amount of one or more anti-tumor agents, such as one selected from the group consisting of: an EGF-receptor antagonist, and arsenic sulfide, adriamycin, cisplatin, carboplatin, cimetidine, caminomycin, mechlorethamine hydrochloride, pentamethylmelamine, thiotepa, teniposide, cyclophosphamide, chlorambucil, demethoxyhypocrellin A, melphalan, ifosfamide, trofosfamide, Treosulfan, podophyllotoxin or podophyllotoxin derivatives, etoposide phosphate, teniposide, etoposide, leurosidine, leurosine, vindesine, 9-aminocamptothecin, camptoirinotecan, crisnatol, Chloroambucil, megestrol, methopterin, mitomycin C, ecteinascidin 743, busulfan, carmustine (BCNU), lomustine (CCNU), lovastatin, 1-methyl-4-phenylpyridinium ion, semustine, staurosporine, streptozocin, thiotepa, phthalocyanine, dacarbazine, aminopterin, methotrexate, trimetrexate, thioguanine, mercaptopurine, fludarabine, pentastatin, cladribin, cytarabine (ara C), porfiromycin, 5-fluorouracil, 6-mercaptopurine, doxorubicin hydrochloride, leucovorin, mycophenoloc acid, daunorubicin, deferoxamine, floxuridine, doxifluridine, ratitrexed, idarubicin, epirubican, pirarubican, zorubicin, mitoxantrone, bleomycin sulfate, mitomycin C, actinomycin D, safracins, saframycins, quinocarcins, discodermolides, vincristine, vinblastine, vinorelbine tartrate, vertoporfin, paclitaxel, tamoxifen, raloxifene, tiazofuran, thioguanine, ribavirin, EICAR, estramustine, estramustine phosphate sodium, flutamide, bicalutamide, buserelin, leuprolide, pteridines, diyneses, levamisole, aflacon, interferon, interleukins, aldesleukin, filgrastim, sargramostim, rituximab, BCG, tretinoin, irinotecan hydrochloride, betamethosone, gemcitabine hydrochloride, verapamil, VP-16, altretamine, thapsigargin and topotecan.

In certain embodiments, the anti-cancer agent induces HIF-1α-dependent transcription.

In certain embodiments, the anti-cancer agent may induce expression of one or more of cyclin G2, IGF2, IGF-BP1, IGF-BP2, IGF-BP3, EGF, WAF-1, TGF-α, TGF-β3, ADM, EPO, IGF2, EG-VEGF, VEGF, NOS2, LEP, LRP1, HK1, HK2, AMF/GP1, ENO1, GLUT1, GAPDH, LDHA, PFKBF₃, PKFL, MIC1, NIP3, NIX and/or RTP801.

In certain embodiments, the anti-cancer agent may induce mitochondrial dysfunction and/or caspase activation.

In certain embodiments, the anti-cancer agent may induce cell cycle arrest at G2/M in the absence of the cardiac glycoside.

In certain embodiments, the anti-cancer agent may be an inhibitor of chromatin function.

In certain embodiments, the anti-cancer agent may be a DNA topoisomerase inhibitor, such as one selected from: adriamycin, amsacrine, camptothecin, daunorubicin, dactinomycin, doxorubicin, eniposide, epirubicin, etoposide, idarubicin, irinotecan (CPT-11) or mitoxantrone.

In certain embodiments, the anti-cancer agent may be a microtubule inhibiting drug, such as a taxane, including paclitaxel, docetaxel, vincristin, vinblastin, nocodazole, epothilones and navelbine.

In certain embodiments, the anti-cancer agent may be a DNA damaging agent, such as actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide or etoposide (VP16).

In certain embodiments, the anti-cancer agent may be an antimetabolite, such as a folate antagonists, or a nucleoside analog. Exemplary nucleoside analogs include pyrimidine analogs, such as 5-fluorouracil; cytosine arabinoside, and azacitidine. In other embodiments, the nucleoside analog is a purine analog, such as 6-mercaptopurine; azathioprine; 5-iodo-2′-deoxyuridine; 6-thioguanine; 2-deoxycoformycin, cladribine, cytarabine, fludarabine, mercaptopurine, thioguanine, and pentostatin. In certain embodiments, the nucleoside analog is selected from AZT (zidovudine); ACV; valacylovir; famiciclovir; acyclovir; cidofovir; penciclovir; ganciclovir; Ribavirin; ddC; ddI (zalcitabine); lamuvidine; Abacavir; Adefovir; Didanosine; d4T (stavudine); 3TC; BW 1592; PMEA/bis-POM PMEA; ddT, HPMPC, HPMPG, HPMPA, PMEA, PMEG, dOTC; DAPD; Ara-AC, pentostatin; dihydro-5-azacytidine; tiazofurin; sangivamycin; Ara-A (vidarabine); 6-MMPR; 5-FUDR (floxuridine); cytarabine (Ara-C; cytosine arabinoside); 5-azacytidine (azacitidine); HBG [9-(4-hydroxybutyl)guanine], (1S,4R)-4-[2-amino-6-cyclopropyl-amino)-9H-purin-9-yl]-2-cyclopentene-1-methanol succinate (“159U89”), uridine; thymidine; idoxuridine; 3-deazauridine; cyclocytidine; dihydro-5-azacytidine; triciribine, ribavirin, and fludrabine.

In certain embodiments, the nucleoside analog is a phosphate ester selected from the group consisting of: Acyclovir; 1-β-D-arabinofuranosyl-E-5-(2-bromovinyl)uracil; 2′-fluorocarbocyclic-2′-deoxyguanosine; 6′-fluorocarbocyclic-2′-deoxyguanosine; 1-(β-D-arabinofuranosyl)-5(E)-(2-iodovinyl)uracil; {(1r-1α,2β,3α)-2-amino-9-(2,3-bis(hydroxymethyl)cyclobutyl)-6H-purin-6-one}Lobucavir; 9H-purin-2-amine, 9-((2-(1-methylethoxy)-1-((1-methylethoxy)methyl)ethoxy)methyl)-(9Cl); trifluorothymidine; 9->(1,3-dihydroxy-2-propoxy)methylguanine (ganciclovir); 5-ethyl-2′-deoxyuridine; E-5-(2-bromovinyl)-2′-deoxyuridine; 5-(2-chloroethyl)-2′-deoxyuridine; buciclovir; 6-deoxyacyclovir; 9-(4-hydroxy-3-hydroxymethylbut-1-yl)guanine; E-5-(2-iodovinyl)-2′-deoxyuridine; 5-vinyl-1-β-D-arabinofuranosyluracil; 1-β-D-arabinofuranosylthymine; 2′-nor-2′-deoxyguanosine; and 1-β-D-arabinofuranosyladenine.

In certain embodiments, the nucleoside analog modulates intracellular CTP and/or dCTP metabolism.

In certain preferred embodiments, the nucleoside analog is gemcitabine.

In certain embodiments, the anti-cancer agent is a DNA synthesis inhibitor, such as a thymidilate synthase inhibitors (such as 5-fluorouracil), a dihydrofolate reductase inhibitor (such as methoxtrexate), or a DNA polymerase inhibitor (such as fludarabine).

In certain embodiments, the anti-cancer agent is a DNA binding agent, such as an intercalating agent.

In certain embodiments, the anti-cancer agent is a DNA repair inhibitor.

In certain embodiments, the anti-cancer agent is part of a combinatorial therapy selected from ABV, ABVD, AC (Breast), AC (Sarcoma), AC (Neuroblastoma), ACE, ACe, AD, AP, ARAC-DNR, B-CAVe, BCVPP, BEACOPP, BEP, BIP, BOMP, CA, CABO, CAF, CAL-G, CAMP, CAP, CaT, CAV, CAVE ADD, CA-VP16, CC, CDDP/VP-16, CEF, CEPP(B), CEV, CF, CHAP, ChlVPP, CHOP, CHOP-BLEO, CISCA, CLD-BOMP, CMF, CMFP, CMFVP, CMV, CNF, CNOP, COB, CODE, COMLA, COMP, Cooper Regimen, COP, COPE, COPP, CP-Chronic Lymphocytic Leukemia, CP-Ovarian Cancer, CT, CVD, CV1, CVP, CVPP, CYVADIC, DA, DAT, DAV, DCT, DHAP, DI, DTIC/Tamoxifen, DVP, EAP, EC, EFP, ELF, EMA 86, EP, EVA, FAC, FAM, FAMTX, FAP, F-CL, FEC, FED, FL, FZ, HDMTX, Hexa-CAF, ICE-T, IDMTX/6-MP, IE, IfoVP, IPA, M-2, MAC-III, MACC, MACOP-B, MAID, m-BACOD, MBC, MC, MF, MICE, MINE, mini-BEAM, MOBP, MOP, MOPP, MOPP/ABV, MP-multiple myeloma, MP-prostate cancer, MTX/6-MO, MTX/6-MP/VP, MTX-CDDPAdr, MV-breast cancer, MV-acute myelocytic leukemia, M-VAC Methotrexate, MVP Mitomycin, MVPP, NFL, NOVP, OPA, OPPA, PAC, PAC-I, PA-CI, PC, PCV, PE, PFL, POC, ProMACE, ProMACE/cytaBOM, PRoMACE/MOPP, Pt/VM, PVA, PVB, PVDA, SMF, TAD, TCF, TIP, TTT, Topo/CTX, VAB-6, VAC, VACAdr, VAD, VATH, VBAP, VBCMP, VC, VCAP, VD, VelP, VIP, VM, VMCP, VP, V-TAD, 5+2, 7+3, “8 in 1.”

In certain embodiments, the anti-cancer agent is selected from altretamine, aminoglutethimide, amsacrine, anastrozole, asparaginase, bcg, bicalutamide, bleomycin, buserelin, busulfan, calcium folinate, campothecin, capecitabine, carboplatin, carmustine, chlorambucil, cisplatin, cladribine, clodronate, colchicine, crisantaspase, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

In certain embodiments, the anti-cancer agent is selected from tamoxifen, 4-(3-chloro-4-fluorophenylamino)-7-methoxy-6-(3-(4-α-morpholinyl)propoxy)quinazoline, 4-(3-ethynylphenylamino)-6,7-bis(2-methoxyethoxy)quinazoline, hormones, steroids, steroid synthetic analogs, 17a-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, chlorotrianisene, hydroxyprogesterone, aminoglutethimide, estramustine, medroxyprogesteroneacetate, leuprolide, flutamide, toremifene, Zoladex, antiangiogenics, matrix metalloproteinase inhibitors, VEGF inhibitors, ZD6474, SU6668, SU11248, anti-Her-2 antibodies (ZD1839 and OS1774), EGFR inhibitors, EKB-569, Imclone antibody C225, src inhibitors, bicalutamide, epidermal growth factor inhibitors, Her-2 inhibitors, MEK-1 kinase inhibitors, MAPK kinase inhibitors, P13 inhibitors, PDGF inhibitors, combretastatins, MET kinase inhibitors, MAP kinase inhibitors, inhibitors of non-receptor and receptor tyrosine kinases (imatinib), inhibitors of integrin signaling, and inhibitors of insulin-like growth factor receptors.

In certain embodiments, the subject combinations are used to inhibit growth of a tumor cell selected from a pancreatic tumor cell, lung tumor cell, a prostate tumor cell, a breast tumor cell, a colon tumor cell, a liver tumor cell, a brain tumor cell, a kidney tumor cell, a skin tumor cell, an ovarian tumor cell and a leukemic blood cell.

In certain embodiments, the subject combination is used in the treatment of a proliferative disorder selected from renal cell cancer, Kaposi's sarcoma, chronic lymphocytic leukemia, lymphoma, mesothelioma, breast cancer, sarcoma, ovarian carcinoma, rectal cancer, throat cancer, melanoma, colon cancer, bladder cancer, mastocytoma, lung cancer, liver cancer, mammary adenocarcinoma, pharyngeal squamous cell carcinoma, prostate cancer, pancreatic cancer, gastrointestinal cancer, and stomach cancer.

In certain embodiments, the refractory cancer is a solid tumor.

In certain embodiments, the solid tumor is a tumor in the pancreas, lung, kidney, ovarian, breast, prostate, stomach, colon, bladder, prostate, brain, skin, testicles, cervix, uterine, bone, or liver.

In certain embodiments, the solid tumor is a pancreatic tumor refractory to treatment by one or more of: fluorouracil, carmustine (BCNU), temozolomide (TMZ), streptozotocin, and gemcitabine.

In certain embodiments, the solid tumor is a lung tumor refractory to etoposide or platinum-based therapy.

In certain embodiments, the lung tumor is refractory small cell lung cancer.

In certain embodiments, the lung tumor is refractory non-small cell lung cancer.

In certain embodiments, the refractory cancer is a hematological cancer.

It is contemplated that all embodiments of the invention may be combined with any other embodiment(s) of the invention.

### BRIEF DESCRIPTION OF THE DRAWINGS

FIG. 1. Schematic diagram of using Sentinel Line promoter-less trap vectors to generate active genetic sites expressing drug selection markers and/or reporters.

FIG. 2. Schematic diagram of creating a Sentinel Line by sequential isolation of cells resistant to positive and negative selection drugs.

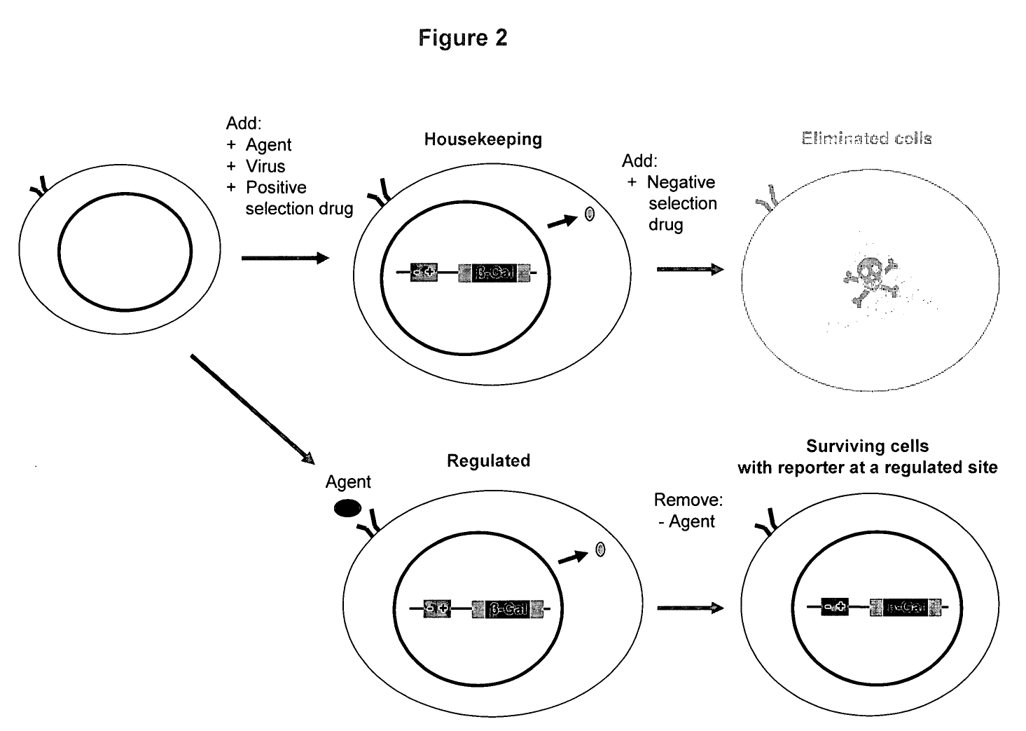

FIG. 3. Adaptation of a cancer cell to hypoxia, which leads to activation of multiple survival factors. The HIF family acts as a master switch transcriptionally activating many genes and enabling factors necessary for glycolytic energy metabolism, angiogenesis, cell survival and proliferation, and erythropoiesis. The level of HIF proteins present in the cell is regulated by the rate of their synthesis in response to factors such as hypoxia, growth factors, androgens and others. Degradation of HIF depends in part on levels of reactive oxygen species (ROS) in the cell. ROS leads to ubiquitylation and degradation of HIF.

FIG. 4. FACS Analysis of Sentinel Lines. Sentinel Lines were developed by transfecting A549 (NSCLC lung cancer) and Panc-1 (pancreatic cancer) cell lines with gene-trap vectors containing E. coli LacZ-encoded β-galactosidase (β-gal) as the reporter gene. The β-gal activity in Sentinel Lines (green) was measured by flow cytometry using a fluorogenic substrate fluoresescein di-beta-D-galactopyranoside (FDG). The auto-fluorescence of untransfected control cells is shown in purple. The graphs indicate frequency of cells (y-axis) and intensity of fluorescence (x-axis) in log scale. The bar charts on the right depict median fluorescent units of the FACS curves. They indicate a high level of reporter activity at the targeted site.

FIG. 5. Western Blot analysis of HIF-1α expression indicates that cardiac glycoside compounds inhibit HIF-1α expression.

FIG. 6. Demonstrates that BNC-1 inhibits HIF-1α synthesis.

FIG. 7. Demonstrates that BNC-1 induces ROS production and inhibits HIF-1α induction in tumor cells.

FIG. 8. Demonstrates that the cardiac glycoside compounds BNC-1 and BNC-4 directly or indirectly inhibits in tumor cells the secretion of the angiogenesis factor VEGF.

FIG. 9. These four charts show FACS analysis of response of a NSCLC Sentinel Line (A549), when treated 40 hrs with four indicated agents. Control (untreated) is shown in purple. Arrow pointing to the right indicates increase in reporter activity whereas inhibitory effect is indicated by arrow pointing to the left. The results indicate that standard chemotherapy drugs turn on survival response in tumor cells.

FIG. 10. Effect of BNC-4 on Gemcitabine-induced stress responses visualized by A549 Sentinel Lines™.

FIG. 11. Pharmacokinetic analysis of BNC-1 delivered by osmotic pumps. Osmotic pumps (Model 2002, Alzet Inc) containing 200 μl of BNC-1 at 50, 30 or 20 mg/ml in 50% DMSO were implanted subcutaneously into nude mice. Mice were sacrificed after 24, 48 or 168 hrs, and plasma was extracted and analyzed for BNC-1 by LC-MS. The values shown are average of 3 animals per point.

FIG. 12. Shows effect of BNC-1 alone or in combination with standard chemotherapy on growth of xenografted human pancreatic tumors in nude mice.

FIG. 13. Shows anti-tumor activity of BNC-1 and Cytoxan against Caki-1 human renal cancer xenograft.

FIG. 14. Shows anti-tumor activity of BNC-1 alone or in combination with Carboplatin in A549 human non-small-cell-lung carcinoma.

FIG. 15. Titration of BNC-1 to determine minimum effective dose effective against Panc-1 human pancreatic xenograft in nude mice. BNC-1 (sc, osmotic pumps) was tested at 10, 5 and 2 mg/ml.

FIG. 16. Combination of BNC-1 with Gemcitabine is more effective than either drug alone against Panc-1 xenografts.

FIG. 17. Combination of BNC-1 with 5-FU is more effective than either drug alone against Panc-1 xenografts.

FIG. 18. Comparison of BNC-1 and BNC-4 in inhibiting hypoxia-mediated HIF-1α induction in human tumor cells (Hep3B cells).

FIG. 19. Comparison of BNC-1 and BNC-4 in inhibiting hypoxia-mediated HIF-1α induction in human tumor cells (Caki-1 and Panc-1 cells).

FIG. 20. BNC-4 blocks HIF-1α induction by a prolyl-hydroxylase inhibitor under normoxia.

FIG. 21. Results showing that the Bufadienolides are more potent Na⁺/K⁺-ATPase inhibitors and cell proliferation inhibitors than the Cardenolides.

FIG. 22. Results showing that BNC-4 alone can significantly reduce tumor growth in xenografted Panc-1 tumors in nude mice.

FIG. 23. Results showing pharmacokinetic analysis of BNC-4 delivered by osmotic pump, and that BNC-4 alone can significantly reduce tumor growth in xenografted Caki-1 human renal tumors in nude mice.

### DETAILED DESCRIPTION OF THE INVENTION

### I. Overview

The present invention is based in part on the discovery that Na⁺/K⁺-ATPase inhibitors, such as cardiac glycosides (e.g., bufadienolides or cardenolides), can be used to effectively treat at least certain cancers refractory to conventional chemo- or radio-therapy.

In a preferred embodiment, the Na⁺/K⁺-ATPase inhibitors are formulated as oral dosage forms, for either single dose or multiple doses per day administration to patients in need thereof.

### II. Definitions

As used herein the term “animal” refers to mammals, preferably mammals such as humans. Likewise, a “patient” or “subject” to be treated by the method of the invention can mean either a human or non-human animal.

As used herein, the term “cancer” refers to any neoplastic disorder, including such cellular disorders as, for example, renal cell cancer, Kaposi's sarcoma, chronic leukemia, prostate cancer, breast cancer, sarcoma, pancreatic cancer, ovarian carcinoma, rectal cancer, throat cancer, melanoma, colon cancer, bladder cancer, mastocytoma, lung cancer, mammary adenocarcinoma, myeloma, lymphoma, pharyngeal squamous cell carcinoma, and gastrointestinal or stomach cancer. Preferably, the cancer which is treated in the present invention is melanoma, lung cancer, breast cancer, pancreatic cancer, prostate cancer, colon cancer, or ovarian cancer.

The “growth state” of a cell refers to the rate of proliferation of the cell and the state of differentiation of the cell.

As used herein, “hyper-proliferative disease” or “hyper-proliferative disorder” refers to any disorder which is caused by or is manifested by unwanted proliferation of cells in a patient. Hyper-proliferative disorders include but are not limited to cancer, psoriasis, rheumatoid arthritis, lamellar ichthyosis, epidermolytic hyperkeratosis, restenosis, endometriosis, and abnormal wound healing.

As used herein, “proliferating” and “proliferation” refer to cells undergoing mitosis.

As used herein, “unwanted proliferation” means cell division and growth that is not part of normal cellular turnover, metabolism, growth, or propagation of the whole organism. Unwanted proliferation of cells is seen in tumors and other pathological proliferation of cells, does not serve normal function, and for the most part will continue unbridled at a growth rate exceeding that of cells of a normal tissue in the absence of outside intervention. A pathological state that ensues because of the unwanted proliferation of cells is referred herein as a “hyper-proliferative disease” or “hyper-proliferative disorder.”

As used herein, “transformed cells” refers to cells that have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. For purposes of this invention, the terms “transformed phenotype of malignant mammalian cells” and “transformed phenotype” are intended to encompass, but not be limited to, any of the following phenotypic traits associated with cellular transformation of mammalian cells: immortalization, morphological or growth transformation, and tumorigenicity, as detected by prolonged growth in cell culture, growth in semi-solid media, or tumorigenic growth in immuno-incompetent or syngeneic animals.

### III. Exemplary Embodiments

Many Na⁺/K⁺-ATPase inhibitors are available in the literature. See, for example, U.S. Pat. No. 5,240,714 which describes a non-digoxin-like Na⁺/K⁺-ATPase inhibitory factor. Recent evidence suggests the existence of several endogenous Na⁺/K⁺-ATPase inhibitors in mammals and animals. For instance, marinobufagenin (3,5-dihydroxy-14,15-epoxy bufodienolide) may be useful in the current combinatorial therapies.

Those skilled in the art can also rely on screening assays to identify compounds that have Na⁺/K⁺-ATPase inhibitory activity. PCT Publications WO00/44931 and WO02/42842, for example, teach high-throughput screening assays for modulators of Na⁺/K⁺-ATPases.

The Na⁺/K⁺-ATPase consists of at least two dissimilar subunits, the large α subunit with all known catalytic functions and the smaller glycosylated β subunit with chaperonic function. In addition there may be a small regulatory, so-called FXYD-peptide. Four a peptide isoforms are known and isoform-specific differences in ATP, Na⁺ and K⁺ affinities and in Ca²⁺ sensitivity have been described. The alpha 1 isoform has been shown to be ubiquitously expressed in all cell types, while the alpha 2 and alpha 3 isoforms are expressed in more excitable tissues such as heart, muscle and CNS. Thus changes in Na⁺/K⁺-ATPase isoform distribution in different tissues, as a function of age and development, electrolytes, hormonal conditions etc. may have important physiological implications. Cardiac glycosides like ouabain are specific inhibitors of the Na⁺/K⁺-ATPase. The four a peptide isoforms have similar high ouabain affinities with K_{d} of around 1 nM or less in almost all mammalian species. In certain embodiments, the Na⁺/K⁺-ATPase inhibitor is more selective for complexes expressed in non-cardiac tissue, relative to cardiac tissue. The following section describes a preferred embodiments of Na⁺/K⁺-ATPase inhibitors-cardiac glycosides.

A. Exemplary Cardiac Glycosides

The subject cardiac glycosides are effective in treating refractory cancers. For example, cardiac glycosides are effective in suppressing EGF, insulin and/or IGF-responsive gene expression in various growth factor responsive cancer cell lines. As another example, the inventors have observed that cardiac glycosides are effective in suppressing HIF-responsive gene expression in cancer cell lines and furthermore, cardiac glycosides are shown to have potent anti-proliferative effects in cancer cell lines. Since Hypoxia appears to promote tumor growth by promoting cell survival through its induction of angiogenesis and its activation of anaerobic metabolism. The inventors have discovered that certain anti-tumor agents in fact promote an hypoxic stress response in tumor cells, which accordingly should have a direct consequence on clinical and prognostic parameters and create a therapeutic challenge. This hypoxic response includes induction of HIF-1 dependent transcription. The effect of HIF-1 on tumor growth is complex and involves the activation of several adaptive pathways. Therefore, hypoxia response of cancer cells in response to certain cancer treatments is at least partially responsible for refractory cancers.

The term “cardiac glycoside” or “cardiac steroid” is used in the medical field to refer to a category of compounds tending to have positive inotropic effects on the heart. As a general class of compounds, cardiac glycosides comprise a steroid core with either a pyrone or butenolide substituent at C17 (the “pyrone form” and “butenolide form”). Additionally, cardiac glycosides may optionally be glycosylated at C3. The form of cardiac glycosides without glycosylation is also known as “aglycone.” Most cardiac glycosides include one to four sugars attached to the 3β-OH group. The sugars most commonly used include L-rhamnose, D-glucose, D-digitoxose, D-digitalose, D-digginose, D-sarmentose, L-vallarose, and D-fructose. In general, the sugars affect the pharmacokinetics of a cardiac glycoside with little other effect on biological activity. For this reason, aglycone forms of cardiac glycosides are available and are intended to be encompassed by the term “cardiac glycoside” as used herein. The pharmacokinetics of a cardiac glycoside may be adjusted by adjusting the hydrophobicity of the molecule, with increasing hydrophobicity tending to result in greater absorption and an increased half-life. Sugar moieties may be modified with one or more groups, such as an acetyl group.

A large number of cardiac glycosides are known in the art for the purpose of treating cardiovascular disorders. Given the significant number of cardiac glycosides that have proven to have anticancer effects in the assays disclosed herein, it is expected that most or all of the cardiac glycosides used for the treatment of cardiovascular disorders may also be used for treating proliferative disorders. Examples of preferred cardiac glycosides include ouabain, digitoxigenin, digoxin and lanatoside C. Additional examples of cardiac glycosides include: Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin and calotoxin. Cardiac glycosides may be evaluated for effectiveness in the treatment of cancer by a variety of methods, including, for example: evaluating the effects of a cardiac glycoside on expression of a HIF-responsive gene in a cancer cell line or evaluating the effects of a cardiac glycoside on cancer cell proliferation.

Notably, cardiac glycosides affect proliferation of cancer cell lines at a concentration well below the known toxicity level. The IC₅₀ measured for ouabain across several different cancer cell lines ranged from about 15 nM to about 600 nM, or about 80 nM to about 300 nM. The concentration at which a cardiac glycoside is effective as part of an anti-proliferative treatment may be further decreased by combination with an additional agent that negatively regulates HIF-responsive genes, such as a redox effector or a steroid signal modulator. For example, as shown herein, the concentration at which a cardiac glycoside (e.g. ouabain or proscillaridin) is effective for inhibiting proliferation of cancer cells is decreased 5-fold by combination with a steroid signal modulator (Casodex). Therefore, in certain embodiments, the invention provides combination therapies of cardiac glycosides with, for example, steroid signal modulators and/or redox effectors. Additionally, cardiac glycosides may be combined with radiation therapy, taking advantage of the radio-sensitizing effect that many cardiac glycosides have.

One exemplary cardiac glycoside is proscillaridin, and its corresponding aglycone is scillarenin. Other cardiac glycosides, such as scillaren, may differ only in glycosylation from proscillaridin, and thus have the same aglycone.

Proscillaridin (such as BNC-4) is a natural product from the Squill plant, Urginea (=Scilla) maritima of the Liliaceae family, a.k.a., “Sea Onion.” The plant was used since antiquity against dropsy (Papyrus Ebers, 1554 B.C., see Jarcho S1974, and Stannard J 1974, and historic references cited therein), presumably for its diuretic properties, and is thus one of the oldest drugs in medicine. Toad toxins, whose chemical structure is very similar to that of Proscillaridin, have been used in China under the name of Ch'an Su for several thousand years for similar indications.

Proscillaridin belongs to the class of cardiac glycosides, steroid-like natural products with a characteristic unsaturated lactone ring attached in beta configuration to carbon 17 (C17). Depending on the ring size, one distinguishes cardenolides (5-membered lactone ring with one double bond) and bufadienolides (6-membered lactone ring with two double bonds). Proscillaridin belongs to the bufadienolide group, while the more frequently used glycosides from the Digitalis plant (Digitoxin, Digoxin) are cardenolides.

On carbon 3 (C3), cardiac glycosides carry up to four sugar molecules, of which glucose and rhamnose are the most common (Proscillaridin is a 3-beta rhamnoside). Unlike in the majority of steroids, the junction between the C and D rings is cis in cardiac glycosides. This configuration, as well as an extended, conjugated π-electronic system with an electron-withdrawing (δ⁻) terminus on carbon 17 in beta-configuration, seems to be essential for the cardiac activity of these compounds (see Thomas R, Gray P, Andrews J. 1990).

Botanical sources of proscillaridin are well-known in the art. For example, such information can be found at various websites, such as maltawildplants dot com/LILI/Urginea maritima.html#BOT. The website shows that the concentration of proscillaridin in the dried squill bulb is about 500 ppm, but its close relative, scillaren, is about 10-times more at 6000 ppm. Although these two compounds slightly differ by the sugar side chains, they both have the same aglycone-scillarenin. As a result, one needs only about 1/10 as much raw material to produce a gram of scillarenin as one needs to produce an equal amount of proscillaridin.

According to the invention, the subject compositions (including the Na⁺/K⁺-ATPase inhibitors, e.g., the cardiac glycosides, the bufadienolides, proscillaridin etc.), are preferably formulated in oral dosage forms. The oral dosage forms of the composition may be in a single dose or multi-dose formulation. The single dose form may be better than the multi-dose form in terms of patient compliance, while the multi-dose form may be better than the single dose in terms of avoiding temporary over-dose due to the rapid absorption of certain subject compositions.

The multi-dose formula may comprise 2-3, or 2-4 doses per day, either in equal amounts, or adjusted for different doses for a particular dose (e.g., the first dose in the morning or the last dose before sleep may be a higher dose to compensate for the long intermission over night).

In certain embodiments, the subject Na⁺/K⁺-ATPase inhibitor is proscillaridin. Exemplary dosages of proscillaridin for the subject invention are provided below. The dosages of any other Na⁺/K⁺-ATPase inhibitors may be deduced based on the exemplary proscillaridin doses, taking into consideration their relative effectiveness and toxicity compared to those of proscillaridin.

In certain embodiments, the oral dosage form of proscillaridin, when delivered to an average adult human, achieves and maintains an effective steady state serum concentration of about 10-700 ng/mL, about 30-500 ng/mL, about 40-500 ng/mL, about 50-500 ng/mL, about 50-400 ng/mL, about 50-300 ng/mL, about 50-200 ng/mL, or about 50-100 ng/mL.

In certain embodiments, the lower end of the concentration is about 10-70 ng/mL, about 30-60 ng/mL, or about 40-50 ng/mL.

In certain embodiments, the high end of the concentration is about 70-500 ng/mL, about 100-500 ng/mL, about 300-500 ng/mL, or about 400-500 ng/mL.

To achieve a steady state level of about 50 ng/mL, a daily total dose of about 2-3 mg is administered to the average human patient. Anti-tumor activity of proscillaridin was observed at a steady state serum level of about 50 ng/mL in a xenograft nude mouse model, where greater than 60% TGI (tumor growth inhibition) was observed. Meanwhile, the maximum toxic dose (MTD) in mice corresponds to a serum levels of about 518 (±121) ng/ml of proscillaridin.

Thus in certain embodiments, about 3-10 mg, about 2.25-7.5 mg, about 1-7.5 mg, about 1.5-5 mg, or about 3-5 mg of proscillaridin is administered per day. In certain other embodiments, an initial dose of about 5-10 mg is administered in the first day, and about 1.5-5 mg is administered every day thereafter.

In certain embodiments, the oral dosage form comprises a daily dose of 2-3 times of 1.5 mg cardiac glycoside or an aglycone thereof.

B. Exemplary Anti-Cancer Agents

Although the subject Na⁺/K⁺-ATPase inhibitors (e.g. cardiac glycosides) can be used alone to treat refractory cancers, they can also be used in combination with other pharmaceutical agents. The pharmaceutical agents that may be used in the subject combination therapy with Na⁺/K⁺-ATPase inhibitors (e.g. cardiac glycosides) include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These anti-cancer agents may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); anti-proliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; chromatin disruptors.

These anti-cancer agents are used by itself with an HIF inhibitor, or in combination. Many combinatorial therapies have been developed in prior art, including but not limited to those listed in Table 1.

TABLE 1
Exemplary conventional combination cancer chemotherapy

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, Prednisone |
| BEACOPP | Bleomycin, Etoposide, Doxorubicin, Cyclophosphamide, Vincristine, |
| | Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or without/Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, Doxorubicin, |
| | Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, Vincristine, Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, Cytarabine |
| COMP | Cyclophosphamide, Vincristine, Methotrexate, Prednisone |
| Cooper Regimen | Cyclophosphamide, Methotrexate, Fluorouracil, Vincristine, Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP (Chronic | Chlorambucil, Prednisone |
| lymphocytic | |
| leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, Fluorouracil |
| ICE-T | Ifosfamide, Carboplatin, Etoposide, Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, Melphalan |
| MAC-III | Methotrexate, Leucovorin, Dactinomycin, Cyclophosphamide |
| MACC | Methotrexate, Doxorubicin, Cyclophosphamide, Lomustine |
| MACOP-B | Methotrexate, Leucovorin, Doxorubicin, Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, Doxorubicin, |
| | Bleomycin, Vinblastine |
| MP (multiple | Melphalan, Prednisone |
| myeloma) | |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDP Adr | Methotrexate, Leucovorin, Cisplatin, Doxorubicin |
| MV (breast cancer) | Mitomycin, Vinblastine |
| MV (acute | Mitoxantrone, Etoposide |
| myelocytic | |
| leukemia) | |
| M-VAC | Vinblastine, Doxorubicin, Cisplatin |
| Methotrexate | |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, Cytarabine, |
| | Bleomycin, Vincristine, Methotrexate, Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, Bleomycin, |
| | Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, Bleomycin |
| VAC | Vincristine, Dactinomycin, Cyclophosphamide |
| VACAdr | Vincristine, Cyclophosphamide, Doxorubicin, Dactinomycin, |
| | Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or Mitoxantrone |
| “8 in 1” | Methylprednisolone, Vincristine, Lomustine, Procarbazine, |
| | Hydroxyurea, Cisplatin, Cytarabine, Dacarbazine |

In addition to conventional anti-cancer agents, the agent of the subject method can also be compounds and antisense RNA, RNAi or other polynucleotides to inhibit the expression of the cellular components that contribute to unwanted cellular proliferation that are targets of conventional chemotherapy. Such targets are, merely to illustrate, growth factors, growth factor receptors, cell cycle regulatory proteins, transcription factors, or signal transduction kinases.

The method of present invention is advantageous over combination therapies known in the art because it allows conventional anti-cancer agent to exert greater effect at lower dosage. In preferred embodiment of the present invention, the effective dose (ED₅₀) for a anti-cancer agent or combination of conventional anti-cancer agents when used in combination with a cardiac glycoside is at least 5 fold less than the ED₅₀ for the anti-cancer agent alone. Conversely, the therapeutic index (TI) for such anti-cancer agent or combination of such anti-cancer agent when used in combination with a cardiac glycoside is at least 5 fold greater than the TI for conventional anti-cancer agent regimen alone.

C. Refractory Tumors Treatable by Na⁺/K⁺-ATPase Inhibitors

Cancers or tumors that are resistant or refractory to treatment of a variety of therapeutic agents may benefit from treatment with the methods of the present invention. Preferred tumors are those resistant to chemotherapeutic agents other than the subject compounds disclosed herein. In certain embodiments of the instant invention, the subject compounds may be useful in treating tumors that are refectory to platinum-based chemotherapeutic agents, including carboplatin, cisplatin, oxaliplatin, iproplatin, tetraplatin, lobaplatin, DCP, PLD-147, JM118, JM216, JM335, and satraplatin. Such platinum-based chemotherapeutic agents also include the platinum complexes disclosed in EP 0147926, U.S. Pat. No. 5,072,011, U.S. Pat. Nos. 5,244,919, 5,519,155, 6,503,943 (LA-12/PLD-147), 6,350,737, and WO 01/064696 (DCP). Resistance to these platinum-based compounds can be tested and verified using the methods described in U.S. Ser. No. 60/546,097.

Suitable agents for which the subject compounds are not cross-resistant are described in the following sections, which may be taken as non-limiting examples of “anti-cancer therapeutic agents.”

1. Taxanes

Resistance to taxanes like pacitaxel and docetaxol is a major problem for all chemotherapeutic regimens utilizing these drugs. Taxanes exert their cytotoxic effect by binding to tubulin, thereby causing the formation of unusually stable microtubules. The ensuing mitotic arrest triggers the mitotic spindle checkpoint and results in apoptosis. Other mechanisms that mediate apoptosis through pathways independent of microtubule dysfunction have been described as well, including molecular events triggered by the activation of Cell Division Control-2 (cdc-2) Kinase, phosphorylation of BCL-2 and the induction of interleukin 1β (IL-1β) and tumor necrosis factor-α (TNF-α). Furthermore, taxanes have been shown to also exert anti-tumor activity via other mechanisms than the direct activation of the apoptotic cascade. These mechanisms include decreased production of metalloproteinases and the inhibition of endothelial cell proliferation and motility, with consequent inhibition of angiogenesis.

Thus, one embodiment of the present invention relates to methods of treating patients with tumors resistant to taxanes by administering a subject compound.

By the term “taxane”, it is meant to include any member of the family of terpenes, including, but not limited to paclitaxel (Taxol) and docetaxel (Taxotere), which were derived primarily from the Pacific yew tree, Taxus brevifolia, and which have activity against certain tumors, particularly breast, lung and ovarian tumors (See, for example, Pazdur et al. Cancer Treat Res. 1993.19:351; Bissery et al. Cancer Res. 1991 51:4845). In the methods and packaged pharmaceuticals of the present invention, preferred taxanes are paclitaxel, docetaxel, deoxygenated paclitaxel, TL-139 and their derivatives. See Annu. Rev. Med. 48:353-374 (1997).

The term “paclitaxel” includes both naturally derived and related forms and chemically synthesized compounds or derivatives thereof with antineoplastic properties including deoxygenated paclitaxel compounds such as those described in U.S. Pat. No. 5,440,056, U.S. Pat. No. 4,942,184, which are herein incorporated by reference, and that sold as TAXOL® by Bristol-Myers Oncology. Paclitaxel has been approved for clinical use in the treatment of refractory ovarian cancer in the United States (Markman et al., Yale Journal of Biology and Medicine, 64:583, 1991; McGuire et al., Ann. Intern. Med., 111:273, 1989). It is effective for chemotherapy for several types of neoplasms including breast (Holmes et al., J. Nat. Cancer Inst., 83:1797, 1991) and has been approved for treatment of breast cancer as well. It is a potential candidate for treatment of neoplasms in the skin (Einzig et al., Proc. Am. Soc. Clin. Oncol., 20:46) and head and neck carcinomas (Forastire et al. Sem. Oncol., 20:56, 1990). The compound also shows potential for the treatment of polycystic kidney disease (Woo et al., Nature, 368:750, 1994), lung cancer and malaria. Docetaxel (N-debenzoyl-N-tert-butoxycarbonyl-10-deacetyl paclitaxel) is produced under the trademark TAXOTERE® by Aventis. In addition, other taxanes are described in “Synthesis and Anticancer Activity of Taxol other Derivatives,” D. G. I. Kingston et al., Studies in Organic Chemistry, vol. 26, entitled “New Trends in Natural Products Chemistry” (1986), Atta-urRabman, P. W. le Quesne, Eds. (Elvesier, Amsterdam 1986), pp 219-235 are incorporated herein. Various taxanes are also described in U.S. Pat. No. 6,380,405, the entirety of which is incorporated herein.

Methods and packaged pharmaceuticals of the present invention are applicable for treating tumors resistant to treatment by any taxane, regardless of the resistance mechanism. Known mechanisms that confer taxane resistance include, for example, molecular changes in the target molecules, i.e., α-tubulin and/or β-tubulin, up-regulation of P-glycoprotein (multidrug resistance gene MDR-1), changes in apoptotic regulatory and mitosis checkpoint proteins, changes in cell membranes, overexpression of interleukin 6 (IL-6; Clin Cancer Res (1999) 5, 3445-3453; Cytokine (2002) 17, 234-242), the overexpression of interleukin 8 (IL-8; Clin Cancer Res (1999) 5, 3445-3453; Cancer Res (1996) 56, 1303-1308) or the overexpression of monocyte chemotactic protein-1 (MCP-1; (MCP-1; Clin Cancer Res (1999) 5, 3445-3453), changes in the levels of acidic and basic fibroblast growth factors, transmembrane factors, such as p185 (HER2; Oncogene (1996) 13, 1359-1365) or EGFR (Oncogene (2000) 19, 6550-6565; Bioessays (2000) 22, 673-680), changes in adhesion molecules, such as β1 integrin (Oncogene (2001) 20, 4995-5004), changes in house keeping molecules, such as glutathione-S-transferase and/or glutathione peroxidase (Jpn J Clin Oncol (1996) 26, 1-5), changes in molecules involved in cell signaling, such as interferon response factor 9, molecules involved in NF-κB signaling, molecules involved in the PI-3 kinase/AKT survival pathway, RAF-1 kinase activity, PKC α/β or PKC β/β2 and via nuclear proteins, such as nuclear annexin IV, the methylation controlled J protein of the DNA J family of proteins, thymidylate synthetase or c-jun.

Another known mechanism that confers taxane resistance is, for example, changes in apoptotic regulatory and mitosis checkpoint proteins. Such changes in apoptotic regulatory and mitosis checkpoint proteins include the over-expression of Bcl-2 (Cancer Chemother Pharmacol (2000) 46, 329-337; Leukemia (1997) 11, 253-257) and the over-expression of Bcl-xL (Cancer Res (1997) 57, 1109-1115; Leukemia (1997) 11, 253-257). Over-expression of Bcl-2 may be effected by estradiol (Breast Cancer Res Treat (1997) 42, 73-81).

Taxane resistance may also be conferred via changes in the cell membrane. Such changes include the change of the ratio of fatty acid methylene:methyl (Cancer Res (1996) 56, 3461-3467), the change of the ratio of choline:methyl (Cancer Res (1996) 56, 3461-3467) and a change of the permeability of the cell membrane (J Cell Biol (1986) 102, 1522-1531).

A further known mechanism that confers taxane resistance is via changes in acidic and basic fibroblast growth factors (Proc Natl Acad Sci USA (2000) 97, 8658-8663), via molecules involved in cell signaling, such as interferon response factor 9 (Cancer Res (2001) 61, 6540-6547), molecules involved in NF-κB signaling (Surgery (2991) 130, 143-150), molecules involved in the PI-3 kinase/AKT survival pathway (Oncogene (2001) 20, 4995-5004), RAF-1 kinase activity (Anticancer Drugs (2000) 11, 439-443; Chemotherapy (2000) 46, 327-334), PKC α/β (Int J Cancer (1993) 54, 302-308) or PKC α/β2 (Int J Cancer (2001) 93, 179-184, Anticancer Drugs (1997) 8, 189-198).

Taxane resistance may also be conferred via changes nuclear proteins, such as nuclear annexin IV (Br J Cancer (2000) 83, 83-88), the methylation controlled J protein of the DNA J family of proteins (Cancer Res (2001) 61, 4258-4265), thymidylate synthetase (Anticancer Drugs (1997) 8, 189-198) or c-jun (Anticancer Drugs (1997) 8, 189-198), via paracrine factors, such as LPS (J Leukoc Biol (1996) 59, 280-286), HIF-1 (Mech Dev (1998) 73, 117-123), VEGF (Mech Dev (1998) 73, 117-123) and the lack of decline in bcl-XL in spheroid cultures (Cancer Res (1997) 57, 2388-2393).

2. Indole Alkaloid

Thus, one embodiment of the present invention relates to methods of treating patients with tumors resistant to an indole alkaloid by administering a subject compound.

Exemplary indole alkaloids include bis-indole alkaloids, such as vincristine, vinblastine and 5′-nor-anhydrovinblastine (hereinafter: 5′-nor-vinblastine). It is known that bis-indole compounds (alkaloids), and particularly vincristine and vinblastine of natural origin as well as the recently synthetically prepared 5′-nor-vinblastine play an important role in the antitumor therapy. These compounds were commercialized or described, respectively in the various pharmacopoeias as salts (mainly as sulfates or difumarates, respectively).

Preferred indole alkaloids are camptothecin and its derivatives and analogues. Camptothecin is a plant alkaloid found in wood, bark, and fruit of the Asian tree Camptotheca acuminata. Camptothecin derivatives are now standard components in the treatment of several malignancies. See Pizzolato and Saltz, 2003. Studies have established that CPT inhibited both DNA and RNA synthesis. Recent research has demonstrated that CPT and CPT analogues interfere with the mechanism of action of the cellular enzyme topoisomerase I, which is important in a number of cellular processes (e.g., DNA replication and recombination, RNA transcription, chromosome decondensation, etc.). Without being bound to theory, camptothecin is thought to reversibly induce single-strand breaks, thereby affecting the cell's capacity to replicate. Camptothecin stabilizes the so-called cleavable complex between topoisomerase I and DNA. These stabilized breaks are fully reversible and non-lethal. However, when a DNA replication fork collides with the cleavable complex, single-strand breaks are converted to irreversible double-strand breaks. Apoptotic cell death is then mediated by caspase activation. Inhibition of caspase activation shifts the cells from apoptosis to transient G1 arrest followed by cell necrosis. Thus, the mechanisms of cell death need active DNA replication to be happening, resulting in cytotoxic effects from camptothecin that is S-phase-specific. Indeed, cells in S-phase in vitro have been shown to be 100-1000 times more sensitive to camptothecin than cells in G1 or G2.

Camptothecin analogues and derivatives include, for example, irinotecan (Camptosar, CPT-11), topotecan (Hycamptin), BAY 38-3441, 9-nitrocamptothecin (Orethecin, rubitecan), exatecan (DX-8951), lurtotecan (GI-147211C), gimatecan, homocamptothecins diflomotecan (BN-80915) and 9-aminocamptothecin (IDEC-13′). See Pizzolato and Saltz, The Lancet, 361:2235-42 (2003); and Ulukan and Swaan, Drug 62: 2039-57 (2002). Additional Camptothecin analogues and derivatives include, SN-38 (this is the active compound of the prodrug irinotecan; conversion is catalyzed by cellular carboxylesterases), ST1481, karanitecin (BNP1350), indolocarbazoles, such as NB-506, protoberberines, intoplicines, idenoisoquinolones, benzo-phenazines and NB-506. More camptothecin derivatives are described in WO03101998: NITROGEN-BASED HOMO-CAMPTOTHECIN DERIVATIVES; U.S. Pat. No. 6,100,273: Water Soluble Camptothecin Derivatives, U.S. Pat. No. 5,587,673, Camptothecin Derivatives.

The methods and pharmaceutical compositions of the present invention are useful for treating tumors resistant to any one or more of above-listed drugs.

3. Platinum-Based Therapeutic Agents

In an alternative embodiment, the methods, packaged pharmaceuticals and pharmaceutical compositions of the present invention are useful for treating tumors resistant to platinum-based chemotherapeutic agents.

Such platinum-based chemotherapeutic agents may include: carboplatin, cisplatin, oxaliplatin, iproplatin, tetraplatin, lobaplatin, DCP, PLD-147, JM118, JM216, JM335, and satraplatin. Such platinum-based chemotherapeutic agents also include the platinum complexes disclosed in EP 0147926, U.S. Pat. No. 5,072,011, U.S. Pat. No. 5,244,919, 5,519,155, 6,503,943 (LA-12/PLD-147), 6,350,737, and WO 01/064696 (DCP).

As is understood in the art, the platinum-based chemotherapeutic agents, or platinum coordination complexes, typified by cisplatin [cis-diamminedichloroplatinum (II)] (Reed, 1993, in Cancer, Principles and Practice of Oncology, pp. 390-4001), have been described as “the most important group of agents now in use for cancer treatment”. These agents, used as a part of combination chemotherapy regimens, have been shown to be curative for testicular and ovarian cancers and beneficial for the treatment of lung, bladder and head and neck cancers. DNA damage is believed to be the major determinant of cisplatin cytotoxicity, though this drug also induces other types of cellular damage.

In addition to cisplatin, this group of drugs includes carboplatin, which like cisplatin is used clinically, and other platinum-containing drugs that are under development. These compounds are believed to act by the same or very similar mechanisms, so that conclusions drawn from the study of the bases of cisplatin sensitivity and resistance are expected to be valid for other platinum-containing drugs.

Cisplatin is known to form adducts with DNA and to induce interstrand crosslinks. Adduct formation, through an as yet unknown signaling mechanism, is believed to activate some presently unknown cellular enzymes involved in programmed cell death (apoptosis), the process which is believed to be ultimately responsible for cisplatin cytotoxicity (see Eastman, 1990, Cancer Cells 2: 275-2802).

Applicants have demonstrated that the subject compounds are effective in treating resistant tumors in which resistance is mediated through at least one of the following three mechanisms: multidrug resistance, tubulins and topoisomerase I. This section describes these three resistance mechanisms and therapeutic agents for which resistance arises through at least one of these mechanisms. One of skill in the art will understand that tumor cells may be resistant to a chemotherapeutic agent through more than one mechanism. For example, the resistance of tumor cells to paclitaxel may be mediated through via multidrug resistance, or alternatively, via tubulin mutation(s).

In a preferred embodiment, the methods and pharmaceutical compositions of the present invention are useful for treating tumors resistant to certain chemotherapeutic agents.

a. Resistance Mediated Through Tubulins

Microtubules are intracellular filamentous structures present in all eukaryotic cells. As components of different organelles such as mitotic spindles, centrioles, basal bodies, cilia, flagella, axopodia and the cytoskeleton, microtubules are involved in many cellular functions including chromosome movement during mitosis, cell motility, organelle transport, cytokinesis, cell plate formation, maintenance of cell shape and orientation of cell microfibril deposition in developing plant cell walls. The major component of microtubules is tubulin, a protein composed of two subunits called alpha and beta. An important property of tubulin in cells is the ability to undergo polymerization to form microtubules or to depolymerize under appropriate conditions. This process can also occur in vitro using isolated tubulin.

Microtubules play a critical role in cell division as components of the mitotic spindle, an organelle which is involved in distributing chromosomes within the dividing cell precisely between the two daughter nuclei. Various drugs prevent cell division by binding to tubulin or to microtubules. Anticancer drugs acting by this mechanism include the alkaloids vincristine and vinblastine, and the taxane-based compounds paclitaxel and docetaxel {see, for example, E. K. Rowinsky and R. C. Donehower, Pharmacology and Therapeutics, 52, 35-84 (1991)}. Other antitubulin compounds active against mammalian cells include benzimidazoles such as nocodazole and natural products such as colchicine, podophyllotoxin, epithilones, and the combretastatins.

Certain therapeutic agents may exert their activities by, for example, binding to α-tubulin, β-tubulin or both, and/or stabilizing microtubules by preventing their depolymerization. Other modes of activity may include, down regulation of the expression of such tubulin proteins, or binding to and modification of the activity of other proteins involved in the control of expression, activity or function of tubulin.

In one embodiment, the resistance of tumor cells to a therapeutic agent is mediated through tubulin. By “mediated through tubulin”, it is meant to include direct and indirect involvement of tubulin. For example, resistance may arise due to tubulin mutation, a direct involvement of tubulin in the resistance. Alternatively, resistance may arise due to alterations elsewhere in the cell that affect tubulin and/or microtubules. These alterations may be mutations in genes affecting the expression level or pattern of tubulin, or mutations in genes affecting microtubule assembly in general. Mammals express 6 β- and 6 β-tubulin genes, each of which may mediate drug resistance.

Specifically, tubulin-mediated tumor resistance to a therapeutic agent may be conferred via molecular changes in the tubulin molecules. For example, molecular changes include mutations, such as point mutations, deletions or insertions, splice variants or other changes at the gene, message or protein level. In particular embodiments, such molecular changes may reside in amino acids 250-300 of β-tubulin, or may affect nucleotides 810 and/or 1092 of the β-tubulin gene. For example, and without wishing to be limited, the paclitaxel-resistant human ovarian carcinoma cell line 1A9-PTX10 is mutated at amino acid residues β 270 and β 364 of β-tubulin (see Giannakakou et al., 1997). For another example, two epothilone-resistant human cancer cell lines has acquired 1-tubulin mutations at amino acid residues 1274 and 0282, respectively (See Giannakakou et al., 2000). These mutations are thought to affect the binding of the drugs to tubulins. Alternatively, mutations in tubulins that confer drug resistance may also be alterations that affect microtubule assembly. This change in microtubule assembly has been demonstrated to compensate for the effect of drugs by having diminished microtubule assembly compared to wild-type controls (Minotti, A. M., Barlow, S. B., and Cabral, F. (1991) J Biol Chem 266, 3987-3994). It will also be understood by a person skilled in the art that molecular changes in α-tubulin may also confer resistance to certain compounds. WO 00/71752 describes a wide range of molecular changes to tubulin molecules and the resistance to certain chemotherapeutic compounds that such molecular changes may confer on a cell. WO 00/71752, and all references therein, are incorporated in their entirety herein.

Tubulin-mediated tumor resistance to therapeutic agents may also be conferred via alterations of the expression pattern of either α-tubulin or the β-tubulin, or both. For example, several laboratories have provided evidence that changes in the expression of specific β-tubulin genes are associated with paclitaxel resistance in cultured tumor cell lines (Haber, M., Burkhart, C. A., Regl, D. L., Madafiglio, J., Norris, M. D., and Horwitz, S. B. (1995) J Biol. Chem. 270, 31269-75; Jaffrezou, J. P., Durnontet, C., Deny, W. B., Duran, G., Chen, G., Tsuchiya, E., Wilson, L., Jordan, M. A., and Sikic, B. l. (1995) Oncology Res. 7, 517-27; Kavallaris, M., Kuo, D. Y. S., Burkhart, C. A., RegI, D. L., Norris, M. D., Haber, M., and Horwitz, S. B. (1997) J. Clin. Invest. 100, 1282-93; and Ranganathan, S., Dexter, D. W., Benetatos, C. A., and Hudes, G. R. (1998) Biochin7. Biophys. Acta 1395, 237-245).

Tubulin-mediated tumor resistance to therapeutic agents may also be conferred via an increase of the total tubulin content of the cell, an increase in the α-tubulin content or the expression of different electrophoretic variants of α-tubulin. Furthermore, resistance may be conferred via alterations in the electrophoretic mobility of β-tubulin subunits, overexpression of the Hβ2 tubulin gene, overexpression of the Hβ3 tubulin gene, overexpression of the Hβ4 tubulin gene, overexpression of the Hβ4a tubulin gene or overexpression of the Hβ5 tubulin gene.

Tubulin-mediated tumor resistance to therapeutic agents may also be conferred via post-translational modification of tubulin, such as increased acetylation of α-tubulin (Jpn J Cancer Res (85) 290-297), via proteins that regulate microtubule dynamics by interacting with tubulin dimmers or polymerized microtubules. Such proteins include but are not limited to stathmin (Mol Cell Biol (1999) 19, 2242-2250) and MAP4 (Biochem Pharmacol (2001) 62, 1469-1480).

Exemplary chemotherapeutic agents for which resistance is at least partly mediated through tubulin include, taxanes (paclitaxel, docetaxel and Taxol derivatives), vinca alkaloids (vinblastine, vincristine, vindesine and vinorelbine), epothilones (epothilone A, epothilone B and discodermolide), nocodazole, colchicin, colchicines derivatives, allocolchicine, Halichondrin B, dolstatin 10, maytansine, rhizoxin, thiocolchicine, trityl cysterin, estramustine and nocodazole. See WO 03/099210 and Giannakakou et al., 2000. Additional exemplary chemotherapeutic agents for which resistance is at least partly mediated through tubulin include, colchicine, curacin, combretastatins, cryptophycins, dolastatin, auristatin PHE, symplostatin 1, eleutherobin, halichondrin B, halimide, hemiasterlins, laulimalide, maytansinoids, PC-SPES, peloruside A, resveratrol, S-allylmercaptocysteine (SAMC), spongistatins, taxanes, vitilevuamide, 2-methoxyestradiol (2-ME2), A-289099, A-293620/A-318315, ABT-751/E7010, ANG 600 series, anhydrovinblastine (AVLB), AVE806, bivatuzumab mertansine, BMS-247550, BMS-310705, cantuzumab mertansine, combretastatin, combretastatin A-4 prodrug (CA4P), CP248/CP461, D-24851/D-64131, dolastatin 10, E7389, EP0906, FR182877, HMN-214, huN901-DM1/BB-10901TAP, ILX-651, KOS-862, LY355703, mebendazole, MLN591DM1, My9-6-DM1, NPI-2352 and NPI-2358, Oxi-4503, R440, SB-715992, SDX-103, T67/T607, trastuzumab-DM1, TZT-1027, vinflunine, ZD6126, ZK-EPO.

Resistance to these and other compounds can be tested and verified using the methods described in the Examples. The methods and pharmaceutical compositions of the present invention are useful for treating tumors resistant to any one or more of above-listed agents.

Preferred chemotherapeutic agents for which resistance is at least partly mediated through tubulin are taxanes, including, but not limited to paclitaxel and docetaxel (Taxotere), which were derived primarily from the Pacific yew tree, Taxus brevifolia, and which have activity against certain tumors, particularly breast and ovarian tumors (See, for example, Pazdur et al. Cancer Treat Res. 1993.19:351; Bissery et al. Cancer Res. 1991 51:4845).

b. Resistance Mediated Through Multidrug Resistance

In another embodiment, the resistance of tumor cells to a therapeutic agent is mediated through multidrug resistance. The term “multidrug resistance (MDR)”, as used herein, refers to a specific mechanism that limits the ability of a broad class of hydrophobic, weakly cationic compounds to accumulate in the cell. These compounds have diverse structures and mechanisms of action yet all are affected by this mechanism.

Experimental models demonstrate that multidrug resistance can be caused by increased expression of ATP-binding cassette (ABC) transporters, which function as ATP-dependent efflux pumps. These pumps actively transport a wide array of anti-cancer and cytotoxic drugs out of the cell, in particular natural hydrophobic drugs. In mammals, the superfamily of ABC transporters includes P-glycoprotein (P-gp) transporters (MDR1 and MDR3 genes in human), the MRP subfamily (already composed of six members), and bile salt export protein (ABCB11; Cancer Res (1998) 58, 4160-4167), MDR-3 (Nature Rev Cancer (2002) 2, 48-58), lung resistance protein (LRP) and breast cancer resistant protein (BCRP). See Kondratov et al., 2001 and references therein; Cancer Res (1993) 53, 747-754; J Biol Chem (1995) 270, 31269-31275; Leukemia (1994) 8, 465-475; Biochem Pharmacol (1997) 53, 461-470; Leonard et al (2003), The Oncologist 8:411-424). These proteins can recognize and efflux numerous substrates with diverged chemical structure, including many anticancer drugs. Overexpression of P-gp is the most common cause for MDR. Other causes of MDR have been attributed to changes in topoisomerase II, protein kinase C and specific glutathione transferase enzymes. See Endicott and Ling, 1989.

The methods of the present invention are useful for treating tumors resistant to a therapeutic agent, in which resistance is at least partially due to MDR. In a preferred embodiment, the drug resistance of the tumor is mediated through overexpression of an ABC transporter. In a further preferred embodiment, the drug resistance of the tumor is mediated through the overexpression of P-gp. Numerous mechanisms can lead to overexpression of P-gp, including amplification of the MDR-1 gene (Anticancer Res (2002) 22, 2199-2203), increased transcription of the MDR-1 gene (J Clin Invest (1995) 95, 2205-2214; Cancer Lett (1999) 146, 195-199; Clin Cancer Res (1999) 5, 3445-3453; Anticancer Res (2002) 22, 2199-2203), which may be mediated by transcription factors such as RGP8.5 (Nat Genet. 2001 (27), 23-29), mechanisms involving changes in MDR-1 translational efficiency (Anticancer Res (2002) 22, 2199-2203), mutations in the MDR-1 gene (Cell (1988) 53, 519-529; Proc Natl Acad Sci USA (1991) 88, 7289-7293; Proc Natl Acad Sci USA (1992) 89, 4564-4568) and chromosomal rearrangements involving the MDR-1 gene and resulting in the formation of hybrid genes (J Clin Invest (1997) 99, 1947-1957).

In other embodiments, the methods of the present invention are useful for treating tumors resistant to a therapeutic agent, in which resistance is due to other causes that lead to MDR, including, for example, changes in topoisomerase II, protein kinase C and specific glutathione transferase enzyme.

Therapeutic agents to which resistance is conferred via the action of P-gp include, but is not limited to: vinca alkaloids (e.g., vinblastine), the anthracyclines (e.g., adriamycin, doxorubicin), the epipodophyllotoxins (e.g., etoposide), taxanes (e.g., paclitaxel, docetaxel), antibiotics (e.g., actinomycin D and gramicidin D), antimicrotubule drugs (e.g., colchicine), protein synthesis inhibitors (e.g., puromycin), toxic peptides (e.g., valinomycin), topoisomerase Inhibitors (e.g., topotecan), DNA intercalators (e.g., ethidium bromide) and anti-mitotics. See WO 99/20791. The methods and pharmaceutical compositions of the present invention are useful for treating tumors resistant to any one or more of above-listed drugs.

c. Resistance Mediated Through Topoisomerase I

In a further embodiment, the resistance of tumor cells to a therapeutic agent is mediated through topoisomerase. Exemplary therapeutic agents that belong to this category include those that target topoisomerase, either directly or indirectly.

DNA normally exists as a supercoiled double helix. During replication, it unwinds, with single strands serving as a template for synthesis of new strands. To relieve the torsional stress that develops ahead of the replication fork, transient cleavage of one or both strands of DNA is needed. Without wishing to be bound to any mechanism, it is believed that Topoisomerases facilitate this process as follows: Topoisomerase II causes transient double-stranded breaks, whereas topoisomerase I causes single-strand breaks. This action allows for rotation of the broken strand around the intact strand. Topoisomerase I then re-ligates the broken strand to restore integrity of double-stranded DNA.

In one embodiment, resistance of tumor cells to a therapeutic agent is mediated through topoisomerase. By “mediated through topoisomerase”, it is meant to include direct and indirect involvement of topoisomerase. For example, resistance may arise due to topoisomerase mutation, a direct involvement of topoisomerase in the resistance. Alternatively, resistance may arise due to alterations elsewhere in the cell that affect topoisomerase. These alterations may be mutations in genes affecting the expression level or pattern of topoisomerase, or mutations in genes affecting topoisomerase function or activity in general. In preferred embodiments said topoisomerase is topoisomerase I. In other embodiments said topoisomerase is Topoisomerase II.

Without being bound by theory, compounds that act on topoisomerase I bind to the topoisomerase I-DNA complex in a manner that prevents the relegation of DNA. Topoisomerase I initially covalently interacts with DNA. Topoisomerase I then cleaves a single strand of DNA and forms a covalent intermediate via a phosphodiester linkage between tyrosine-273 of topoisomerase I and the 3′-phosphate group of the scissile strand of DNA. The intact strand of DNA is then passed through the break and then topoisomerase I religates the DNA and releases the complex. Drugs such as camptothecins bind to the covalent complex in a manner that prevents DNA relegation. The persistent DNA breaks induce apoptosis, likely via collisions between these lesions and or replication or transcription complexes.

Preferred therapeutic agents to which resistance is mediated through topoisomerase I include camptothecin and its derivatives and analogues, such as 9-nitrocamptothecin (IDEC-132), exatecan (DX-8951f), rubitecan (9-nitrocamptothecin), lurtotecan (G1-147211C), the homocamptothecins such as diflomotecan (BN-80915) and BN-80927, topotecan, NB-506, J107088, pyrazolo[1,5-a]indole derivatives, such as GS-5, lamellarin D, SN-38, 9-aminocamptothecin, ST1481 and karanitecin (BNP1350) and irinotecan (CPT-11). Other related camptothecins can be found in The Camptothecins: Unfolding Their Anticancer Potential, Annals of the New York Academy of Science, Volume 922 (ISBN 1-57331-291-6).

Without wishing to be bound by any particular theory, it is believed that camptothecins inhibit topoisomerase I by blocking the rejoining step of the cleavage/religation reaction of topoisomerase I, resulting in accumulation of a covalent reaction intermediate, the cleavable complex. Specifically, topoisomerase I-mediated tumor resistance to therapeutic agents may be conferred via molecular changes in the topoisomerase I molecules. For example, molecular changes include mutations, such as point mutations, deletions or insertions, splice variants or other changes at the gene, message or protein level.

In particular embodiments, such molecular changes reside near the catalytic tyrosine residue at amino acid position 723. Residues at which such molecular changes may occur include but are not limited to amino acid positions 717, 722, 723, 725, 726, 727, 729, 736 and 737 (see Oncogene (2003) 22, 7296-7304 for a review).

In equally preferred embodiments, such molecular changes reside between amino acids 361 and 364. Residues at which such molecular changes may occur include but are not limited to amino acid positions 361, 363 and 364.

In other equally preferred embodiments, such molecular changes reside near amino acid 533. Residues at which such molecular changes may occur include but are not limited to amino acid positions 503 and 533.

In other equally preferred embodiments, such molecular changes may also reside in other amino acids of the topoisomerase I protein. Residues at which such molecular changes may occur include but are not limited to amino acid positions 418 and 503.

In other embodiments, such molecular changes may be a duplication. In one embodiment such a duplication may reside in the nucleotides corresponding to amino acids 20-609 of the topoisomerase I protein.

In other embodiments, topoisomerase I-mediated tumor resistance may also be conferred via cellular proteins that interact with topoisomerase-1. Proteins that are able to do so include, but are not limited to, nucleolin.

In particular embodiments, such molecular changes may reside in amino acids 370 and/or 723. For example, and without wishing to be limited, the camptothecin-resistant human leukemia cell line CEM/C2 (ATCC No. CRL-2264) carries two amino acid substitution at positions 370 (Met→Thr) and 722 (Asn→Ser) (Cancer Res (1995) 55, 1339-1346). The camptothecin resistant CEM/C2 cells were derived from the T lymphoblastoid leukemia cell line CCRF/CEM by selection in the presence of camptothecin in vitro (Kapoor et al., 1995. Oncology Research 7; 83-95, ATCC). The CEM/C2 resistant cells display atypical multi-drug resistance and express a form of topoisomerase I that is less sensitive to the inhibitory action of camptothecin than that from CCRF/CEM cells at a reduced level relative to the parental cells. In addition to resistance to camptothecin, the CEM/C2 cells exhibit cross resistance to etoposide, dactinomycin, bleomycin, mitoxantrone, daunorubicin, doxorubicin and 4′-(9-acridinylamino)methanesulfon-m-anisidide.

In other embodiments, topoisomerase I-mediated tumor resistance to therapeutic agents may also be conferred via alterations of the expression pattern the topoisomerase I gene (Oncol Res (1995) 7, 83-95). In further embodiments, topoisomerase 1-mediated tumor resistance may also be conferred via altered metabolism of the drug. In yet further embodiments, topoisomerase I-mediated tumor resistance may also be conferred via inadequate and/or reduced accumulation of drug in the tumor, alterations in the structure or location of topoisomerase 1, alterations in the cellular response to the topoisomerase I-drug interaction or alterations in the cellular response to drug-DNA-ternary complex formation (Oncogene (2003) 22, 7296-7304; Ann NY Acad Sci (2000) 922, 46-55).

Topoisomerase I is believed to move rapidly from the nucleolus to the nucleus or even cytoplasm after cellular exposure to camptothecins.

In one embodiment topoisomerase I-mediated tumor resistance is mediated through factors involved in the relocation of topoisomerase I from the nucleolus to the nucleus and/or the cytoplasm, such as factors involved in the ubiquitin/26S proteasome pathway or SUMO.

In other embodiments topoisomerase I-mediated tumor resistance is mediated through factors involved in DNA replication, DNA checkpoint control and DNA repair.

Factors of the DNA checkpoint control include proteins of the S-checkpoint control, such as Chk1, ATR, ATM, and the DNA-PK multimer.

In other embodiments topoisomerase I-mediated tumor resistance is mediated via factors of apoptosis pathways or other cell death pathways. This includes, but is not limited to, the overexpression of bcl-2 and the overexpression of p21^{Waf1/Cip1}.

In other embodiments topoisomerase I-mediated tumor resistance is mediated via post-translational modifications of topoisomerase I. Such post-translational modifications are ubiquitination and sumoylation. Furthermore, such post-translational modifications may involve other cellular proteins, such as Ubp11, DOA4 and topor.

Therapeutic agents to which resistance is mediated through topoisomerase II include epipodophyllotoxins, such as VP16 and VM26, [1,5-a], pyrazolo[1,5-a]indole derivatives, such as GS-2, GS-3, GS-4 and GS-5.

d. Resistance to Mitoxanthrone

In another embodiment, tumor cells resistant to Mitoxantrone can be treated using the subject compounds.

Resistance to the anticancer drug mitoxantrone has been associated with several mechanisms, including drug accumulation defects and reduction in its target proteins topoisomerase II α and β4. Recently, overexpression of the breast cancer resistance half transporter protein (BCRP1) was found to be responsible for the occurrence of mitoxantrone resistance in a number of cell lines (Ross et al., J. Natl. Cancer Inst. 91: 429-433, 1999; Miyake et al., Cancer Res. 59: 8-13, 1999; Litman et al., J. Cell Sci. 113(Pt 11): 2011-2021, 2000; Doyle et al., Proc. Natl. Acad. Sci. U.S.A. 95: 15665-15670, 1998). However, not all mitoxantrone resistant cell lines express BCRP1 (Hazlehurst et al., Cancer Res. 59: 1021-1028, 1999; Nielsen et al., Biochem. Pharmacol. 60: 363-370, 2000). The efflux pump responsible for the mitoxantrone resistance in these cell lines is less clear. Boonstra et al. (Br J. Cancer. 2004 May 18 [Epub ahead of print]) report that overexpression of the ABC transporter ABCA2 may lead to the efflux of mitoxantrone by exploring estramustine, which is able to block mitoxantrone efflux in the mitoxantrone resistant GLC4 sub line GLC4-MITO (does not overexpress BCRP1).

The methods of the present invention are useful for treating tumors resistant to a mitoxanthrone.

D. Other Treatment Methods

In yet other embodiments, the subject method combines a Na⁺/K⁺-ATPase inhibitor (e.g. cardiac glycoside) with radiation therapies, including ionizing radiation, gamma radiation, or particle beams.

E. Administration

The Na⁺/K⁺-ATPase inhibitor (e.g. cardiac glycoside), or a combination containing a Na⁺/K⁺-ATPase inhibitor (e.g. cardiac glycoside) may be administered orally, parenterally by intravenous injection, transdermally, by pulmonary inhalation, by intravaginal or intrarectal insertion, by subcutaneous implantation, intramuscular injection or by injection directly into an affected tissue, as for example by injection into a tumor site. In some instances the materials may be applied topically at the time surgery is carried out. In another instance the topical administration may be ophthalmic, with direct application of the therapeutic composition to the eye.

In a preferred embodiment, the subject Na⁺/K⁺-ATPase inhibitors (e.g. cardiac glycosides) are administered to a patient by using osmotic pumps, such as Alzet® Model 2002 osmotic pump. Osmotic pumps provides continuous delivery of test agents, thereby eliminating the need for frequent, round-the-clock injections. With sizes small enough even for use in mice or young rats, these implantable pumps have proven invaluable in predictably sustaining compounds at therapeutic levels, avoiding potentially toxic or misleading side effects.

To meet different therapeutic needs, ALZET's osmotic pumps are available in a variety of sizes, pumping rates, and durations. At present, at least ten different pump models are available in three sizes (corresponding to reservoir volumes of 100 μL, 200 μL and 2 mL) with delivery rates between 0.25 μL/hr and 10 L/1 hr and durations between one day to four weeks.

While the pumping rate of each commercial model is fixed at manufacture, the dose of agent delivered can be adjusted by varying the concentration of agent with which each pump is filled. Provided that the animal is of sufficient size, multiple pumps may be implanted simultaneously to achieve higher delivery rates than are attainable with a single pump. For more prolonged delivery, pumps may be serially implanted with no ill effects. Alternatively, larger pumps for larger patients, including human and other non-human mammals may be custom manufactured by scaling up the smaller models.

The materials are formulated to suit the desired route of administration. The formulation may comprise suitable excipients include pharmaceutically acceptable buffers, stabilizers, local anesthetics, and the like that are well known in the art. For parenteral administration, an exemplary formulation may be a sterile solution or suspension; For oral dosage, a syrup, tablet or palatable solution; for topical application, a lotion, cream, spray or ointment; for administration by inhalation, a microcrystalline powder or a solution suitable for nebulization; for intravaginal or intrarectal administration, pessaries, suppositories, creams or foams. Preferably, the route of administration is parenteral, more preferably intravenous.

### EXEMPLIFICATION

The following examples are for illustrative purpose only, and should in no way be construed to be limiting in any respect of the claimed invention.

The exemplary cardiac glycosides used in following studies are referred to as BNC-1 and BNC-4.

BNC-1 is ouabain or g-Strophanthin (STRODIVAL®), which has been used for treating myocardial infarction. It is a colorless crystal with predicted IC₅₀ of about 0.009-0.035 μg/mL and max. plasma concentration of about 0.03 μg/mL. According to the literature, its plasma half-life in human is about 20 hours, with a range of between 5-50 hours. Its common formulation is injectable. The typical dose for current indication (i.v.) is about 0.25 mg, up to 0.5 mg/day.

BNC-4 is proscillaridin (TALUSIN®), which has been approved for treating chronic cardiac insufficiency in Europe. It is a colorless crystal with predicted IC₅₀ of about 0.002-0.008 zg/mL and max. plasma concentration of about 0.1 μg/mL. According to the literature, its plasma half-life in human is about 40 hours. Its common available formulation is a tablet of 0.25 or 0.5 mg. The typical dose for current indication (p.o.) is about 1.5 mg/day.

### Example I

### Sentinel Line Plasmid Construction and Virus Preparation

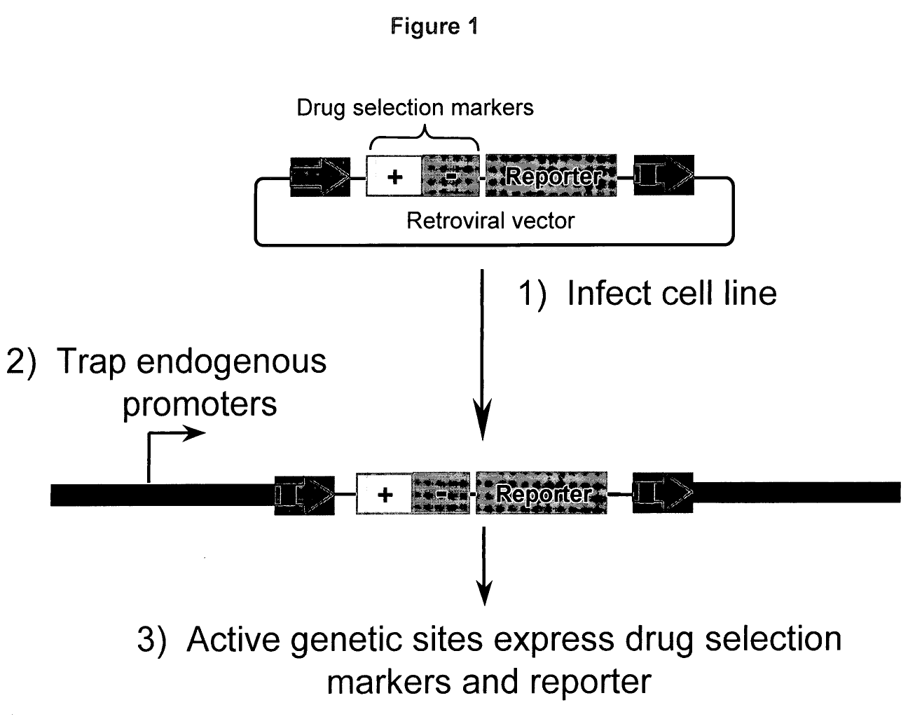

FIG. 1 is a schematic drawing of the Sentinel Line promoter trap system, and its use in identifying regulated genetic sites and in reporting pathway activity. Briefly, the promoter-less selection markers (either positive or negative selection markers, or both) and reporter genes (such as beta-gal) are put in a retroviral vector (or other suitable vectors), which can be used to infect target cells. The randomly inserted retroviral vectors may be so positioned that an active upstream heterologous promoter may initiate the transcription and translation of the selectable markers and reporter gene(s). The expression of such selectable markers and/or reporter genes is indicative of active genetic sites in the particular host cell.

In one exemplary embodiment, the promoter trap vector BV7 was derived from retrovirus vector pQCXIX (BD Biosciences Clontech) by replacing sequence in between packaging signal (Psi⁺) and 3′ LTR with a cassette in an opposite orientation, which contains a splice acceptor sequence derived from mouse engrailed 2 gene (SA/en2), an internal ribosomal entry site (IRES), a LacZ gene, a second IRES, and fusion gene TK:Sh encoding herpes virus thymidine kinase (HSV-tk) and phleomycin followed by a SV40 polyadenylation site. BV7 was constructed by a three-way ligation of three equal molar DNA fragments. Fragment 1 was a 5 kb vector backbone derived from pQCXIX by cutting plasmid DNA extracted from a Dam-bacterial strain with Xho I and Cla I (Dam-bacterial strain was needed here because Cla I is blocked by overlapping Dam methylation). Fragment 2 was a 2.5 kb fragment containing an IRES and a TK:Sh fusion gene derived from plasmid pIREStksh by cutting Dam-plasmid DNA with Cla I and Mlu I. pIREStksh was constructed by cloning TK:Sh fragment from pMODtksh (InvivoGen) into pIRES (BD Biosciences Clontech). Fragment 3 was a 5.8 kb SA/en2-IRES-LacZ fragment derived from plasmid pBSen2IRESLacZ by cutting with BssH II (compatible end to Mlu I) and Xho I. pBSen2IRESLacZ was constructed by cloning IRES fragment from pIRES and LacZ fragment from pMODLacZ (InvivoGen) into plasmid pBSen2.

To prepare virus, packaging cell line 293T was co-transfected with three plasmids BV7, pVSV-G (BD Biosciences Clontech) and pGag-Pol (BD Biosciences Clontech) in equal molar concentrations by using Lipofectamine 2000 (InvitroGen) according to manufacturer's protocol. First virus “soup” (supernatant) was collected 48 hours after transfection, second virus “soup” was collected 24 hours later. Virus particles were pelleted by centrifuging at 25,000 rpm for 2 hours at 4° C. Virus pellets were re-dissolved into DMEM/10% FBS by shaking overnight. Concentrated virus solution was aliquot and used freshly or frozen at −80° C.

### Example II

### Sentinel Line Generation

Target cells were plated in 150 mm tissue culture dishes at a density of about 1×10⁶/plate. The following morning cells were infected with 250 μl of Bionaut Virus #7 (BV7) as prepared in Example 1, and after 48 hr incubation, 20 μg/ml of phleomycin was added. 4 days later, media was changed to a reduced serum (2% FBS) DMEM to allow the cells to rest. 48 h later, ganciclovir (GCV) (0.4 μM, sigma) was added for 4 days (media was refreshed on day 2). One more round of phleomycin selection followed (20 μg/ml phleomycin for 3 days). Upon completion, media was changed to 20% FBS DMEM to facilitate the outgrowths of the clones. 10 days later, clones were picked and expanded for further analysis and screening.

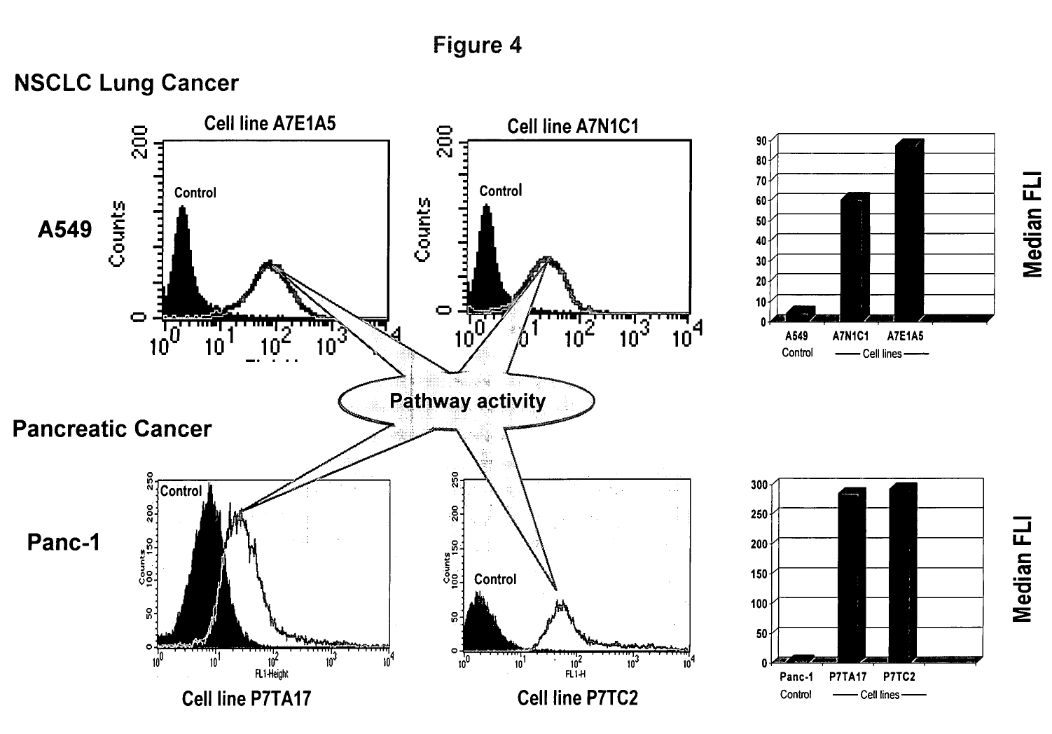

Using this method, several Sentinel Lines were generated to report activity of genetic sites activated by hypoxia pathways (FIG. 4). These Sentinel lines were generated by transfecting A549 (NSCLC lung cancer) and Panc-1 (pancreatic cancer) cell lines with the subject gene-trap vectors containing E. coli LacZ-encoded β-galactosidase (β-gal) as the reporter gene (FIG. 4). The β-gal activity in Sentinel Lines (green) was measured by flow cytometry using a fluorogenic substrate fluoresescein di-beta-D-galactopyranoside (FDG). The autofluorescence of untransfected control cells is shown in purple. The graphs indicate frequency of cells (y-axis) and intensity of fluorescence (x-axis) in log scale. The bar charts on the right depict median fluorescent units of the FACS curves. They indicate a high level of reporter activity at the targeted site.

### Example III

### Cell Culture and Hypoxic Conditions

All cell lines can be purchased from ATCC, or obtained from other sources.

A549 (CCL-185) and Panc-1 (CRL-1469) were cultured in Dulbecco's Modified Eagle's Medium (DMEM), Caki-1 (HTB-46) in McCoy's 5a modified medium, Hep3B (HB-8064) in MEM-Eagle medium in humidified atmosphere containing 5% CO₂ at 37° C. Media was supplemented with 10% FBS (Hyclone; SH30070.03), 100 μg/ml penicillin and 50 μg/ml streptomycin (Hyclone).

To induce hypoxia conditions, cells were placed in a Billups-Rothenberg modular incubator chamber and flushed with artificial atmosphere gas mixture (5% CO₂, 1% O₂, and balance N₂). The hypoxia chamber was then placed in a 37° C. incubator. L-mimosine (Sigma, M-0253) was used to induce hypoxia-like HIF 1-alpha expression. Proteasome inhibitor, MG132 (Calbiochem, 474791), was used to protect the degradation of HIF1-alpha. Cycloheximide (Sigma, 4859) was used to inhibit new protein synthesis of HIF1-alpha. Catalase (Sigma, C3515) was used to inhibit reactive oxygen species (ROS) production.

### Example IV

### Identification of Trapped Genes

Once a Sentinel Line with a desired characteristics was established, it might be helpful to determine the active promoter under which control the markers/reporter genes are expressed. To do so, total RNAs were extracted from cultured Sentinel Line cells by using, for example, RNA-Bee RNA Isolation Reagent (TEL-TEST, Inc.) according to the manufacturer's instructions. Five prime ends of the genes that were disrupted by the trap vector BV7 were amplified by using BD SMART RACE cDNA Amplification Kit (BD Biosciences Clontech) according to the manufacturer's protocol. Briefly, 1 μg total RNA prepared above was reverse-transcribed and extended by using BD PowerScriptase with 5′CDS primer and BD SMART II Oligo both provided by the kit. PCR amplification were carried out by using BD Advantage 2 Polymerase Mix with Universal Primer A Mix provided by the kit and BV7 specific primer 5′Rsa/ires (gacgcggatcttccgggtaccgagctcc, 28 mer). 5′Rsa/ires located in the junction of SA/en2 and IRES with the first 7 nucleotides matching the last 7 nucleotides of SA/en2 in complementary strand. 5′ RACE products were cloned into the TA cloning vector pCR2.1 (InvitroGen) and sequenced. The sequences of the RACE products were analyzed by using the BLAST program to search for homologous sequences in the database of GenBank. Only those hits which contained the transcript part of SA/en2 were considered as trapped genes.

Using this method, the upstream promoters of several Sentinel Lines generated in Example II were identified (see below). The identity of these trapped genes validate the clinical relevance of these Sentinel Lines™, and can be used as biomarkers and surrogate endpoints in clinical trials.

| Sentinel Lines | Genetic Sites | Gene Profile |
|---|---|---|
| A7N1C1 | Essential Antioxidant | Tumor cell-specific gene, over |
| | | expressed in lung tumor cells |
| A7N1C6 | Chr. 3, BAC, map to 3p | novel |
| A7I1C1 | Pyruvate Kinase (PKM | Described biomarker for |
| | 2), Chr. 15 | NSCLC |
| A6E2A4 | 6q14.2-16.1 | Potent angiogenic activity |
| A7I1D1 | Chr. 7, BAC | novel |

### Example V

### Western Blots

For HIF1-alpha Western blots, Hep3B cells were seeded in growth medium at a density of 7×10⁶ cells per 100 mm dish. Following 24-hour incubation, cells were subjected to hypoxic conditions for 4 hours to induce HIF1-alpha expression together with an agent such as 1 μM BNC-1. The cells were harvested and lysed using the Mammalian Cell Lysis kit (Sigma, M-0253). The lysates were centrifuged to clear insoluble debris, and total protein contents were analyzed with BCA protein assay kit (Pierce, 23225). Samples were fractionated on 3-8% Tris-Acetate gel (Invitrogen NUPAGE system) by sodium dodecyl sulfate (SDS)-polyacrylamide gel electropherosis and transferred onto nitrocellulose membrane. HIF1-alpha protein was detected with anti-HIF1-alpha monoclonal antibody (BD Transduction Lab, 610959) at a 1:500 dilution with an overnight incubation at 4° C. in Tris-buffered solution-0.1% Tween 20 (TBST) containing 5% dry non-fat milk. Anti-Beta-actin monoclonal antibody (Abcam, ab6276-100) was used at a 1:5000 dilution with a 30-minute incubation at room temperature. Immunoreactive proteins were detected with stabilized goat-anti mouse HRP conjugated antibody (Pierce, 1858413) at a 1:10,000 dilution. The signal was developed using the West Femto substrate (Pierce, 34095).

We examined the inhibitory effect of BNC-1 on HIF-1 alpha synthesis. 24 hours prior to treatment, Hep3B cells were seeded in growth medium. To show that BNC-1 inhibits HIF1-alpha expression in a concentration dependent manner, cells were treated with 1 μM BNC-1 together with the indicated amount of MG132 under hypoxic conditions for 4 hours. To understand specifically the impact of BNC-1 on HIF-1 alpha synthesis, Hep3B cells were treated with MG132 and 1 μM BNC under normoxic conditions for the indicated time points. The observed expression is accounted by protein synthesis.

We examined the role of BNC-1 on the degradation rate of HIF-1 alpha. 24 hours prior to treatment, Hep3B cells were seeded in growth medium. The cells were placed in hypoxic conditions for 4 hours for HIF1-alpha accumulation. The protein synthesis inhibitor, cycloheximide (100 μM) together with 1 μM BNC-1 were added to the cells and kept in hypoxic conditions for the indicate time points.

To induce HIF1-alpha expression using an iron chelator, L-mimosine was added to Hep3B cells, seeded 24 hours prior, and placed under normoxic conditions for 24 hours.

### Example VI

### Sentinel Line Reporter Assays

The expression level of beta-galactosidase gene in sentinel lines was determined by using a fluorescent substrate fluorescein di-β-D-Galactopyranside (FDG, Marker Gene Tech, #M0250) introduced into cells by hypotonic shock. Cleavage by beta-galactosidase results in the production of free fluorescein, which is unable to cross the plasma membrane and is trapped inside the beta-gal positive cells. Briefly, the cells to be analyzed are trypsinized, and resuspended in PBS containing 2 mM FDG (diluted from a 10 mM stock prepared in 8:1:1 mixture of water:ethanol:DMSO). The cells were then shocked for 4 minutes at 37° C. and transferred to FACS tubes containing cold 1×PBS on ice. Samples were kept on ice for 30 minutes and analyzed by FACS in FL1 channel.

### Example VII

### Testing Standard Chemotherapeutic Agents

Sentinel Line cells with beta-galactosidase reporter gene were plated at 1×10⁵ cells/10 cm dish. After overnight incubation, the cells were treated with standard chemotherapeutic agents, such as mitoxantrone (8 nM), paclitaxel (1.5 nM), carboplatin (15 μM), gemcitabine (2.5 nM), in combination with one or more BNC compounds, such as BNC-1 (10 nM), BNC2 (2 μM), BNC3 (100 μM) and BNC-4 (10 nM), or a targeted drug, Iressa (4 μM). After 40 hrs, the cells were trypsinized and the expression level of reporter gene was determined by FDG loading.

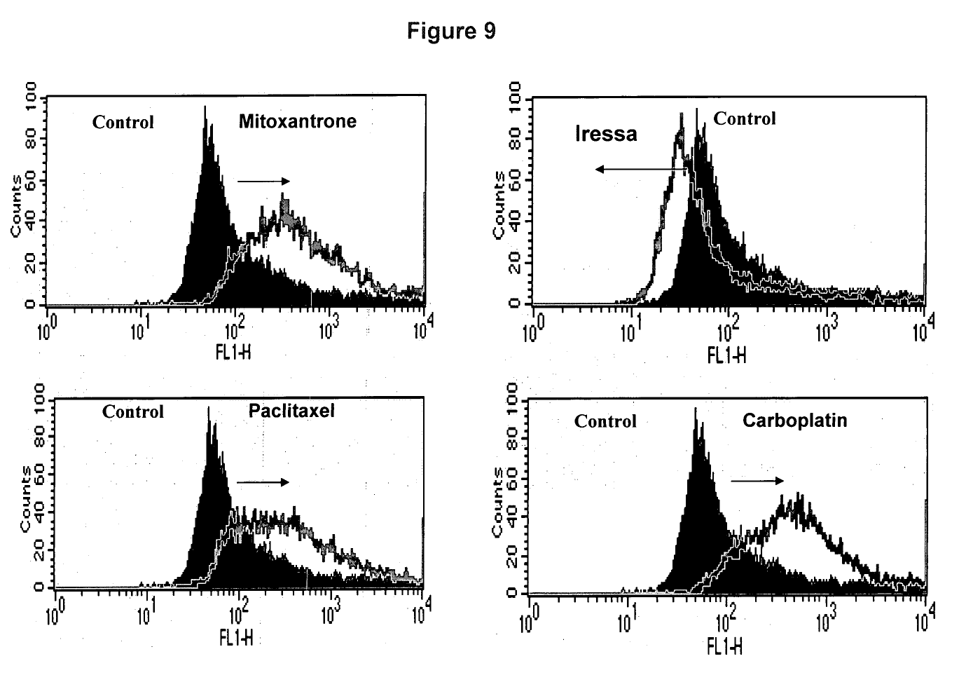

When tested in the Sentinel Lines, mitoxanthrone, paclitaxel, and carboplatin each showed increases in cell death and reporter activity (see FIG. 9). No effect had been expected from the cytotoxic agents because of their nonspecific mechanisms of action (MOA), making their increased reporter activity in HIF-sensitive cell lines surprising. These results provide a previously unexplored link between the development of chemotherapy resistance and induction of the hypoxia response in cells treated with anti-neoplastic agents. Iressa, on the other hand, a known blocker of EGFR-mediated HIF-1 induction, showed a reduction in reporter activity when tested. The Sentinel Lines thus provide a means to differentiate between a cytotoxic agent and a targeted drug.

### Example VIII

### Pharmacokinetic (PK) Analysis

The following protocol can be used to conduct pharmacokinetic analysis of any compounds of the invention. To illustrate, BNC-1 is used as an example.

Nude mice were dosed i.p. with 1, 2, or 4 mg/kg of BNC-1. Venous blood samples were collected by cardiac puncture at the following 8 time points: 5 min, 15 min, 30 min, 45 min, 1 hr, 2 hr, 4 hr, 8 hr, and 24 hr. For continuous BNC-1 treatment, osmotic pumps (such as Alzet® Model 2002) were implanted s.c. between the shoulder blades of each mouse. Blood was collected at 24 hr, 48 hr and 72 hr. Triplicate samples per dose (i.e. three mice per time point per dose) were collected for this experiment.

Approximately 0.100 mL of plasma was collected from each mouse using lithium heparin as anticoagulant. The blood samples were processed for plasma as individual samples (no pooling). The samples were frozen at −70° C. (±10° C.) and transferred on dry ice for analysis by HPLC.

For PK analysis plasma concentrations for each compound at each dose were analyzed by non-compartmental analysis using the software program WinNonlin®. The area under the concentration vs time curve AUC(0-Tf) from time zero to the time of the final quantifiable sample (Tf) was calculated using the linear trapezoid method. AUC is the area under the plasma drug concentration-time curve and is used for the calculation of other PK parameters. The AUC was extrapolated to infinity (0-Inf) by dividing the last measured concentration by the terminal rate constant (k), which was calculated as the slope of the log-linear terminal portion of the plasma concentrations curve using linear regression. The terminal phase half-life (t_{1/2}) was calculated as 0.693/k and systemic clearance (Cl) was calculated as the dose(mg/kg)/AUC(Inf). The volume of distribution at steady-state (Vss) was calculated from the formula:
Vₛₛ=dose(AUMC)/(AUC)²

where AUMC is the area under the first moment curve (concentration multiplied by time versus time plot) and AUC is the area under the concentration versus time curve. The observed maximum plasma concentration (Cₘₐₓ) was obtained by inspection of the concentration curve, and Tₘₐₓ is the time at when the maximum concentration occurred.

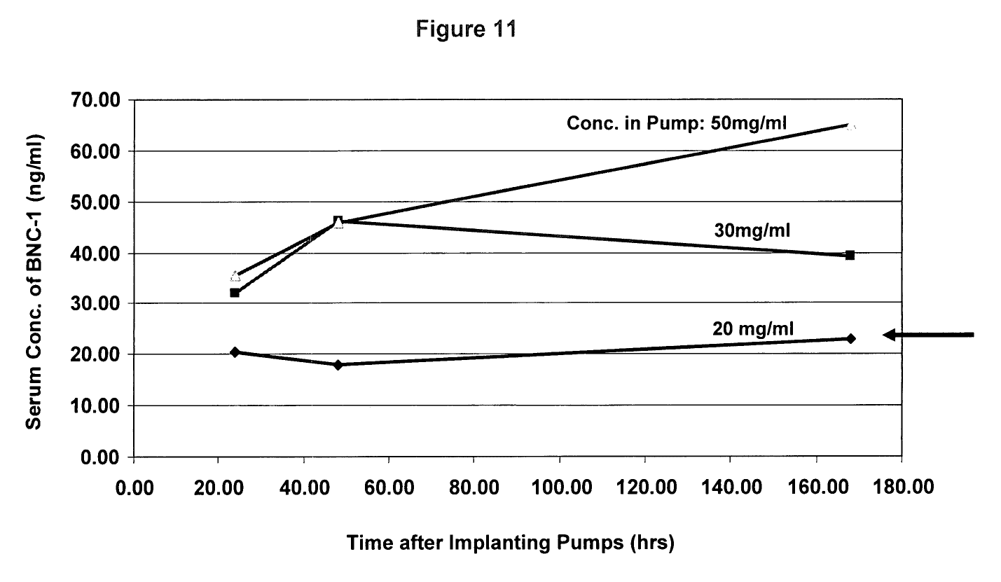

FIG. 11 shows the result of a representative pharmacokinetic analysis of BNC-1 delivered by osmotic pumps. Osmotic pumps (Model 2002, Alzet Inc) containing 200 μl of BNC-1 at 50, 30 or 20 mg/ml in 50% DMSO were implanted subcutaneously into nude mice. Mice were sacrificed after 24, 48 or 168 hrs, and plasma was extracted and analyzed for BNC-1 by LC-MS. The values shown are average of 3 animals per point.

### Example IX

### Human Tumor Xenograft Models

Female nude mice (nu/nu) between 5 and 6 weeks of age weighing approximately 20 g were implanted subcutaneously (s.c.) by trocar with fragments of human tumors harvested from s.c. grown tumors in nude mice hosts. When the tumors were approximately 60-75 mg in size (about 10-15 days following inoculation), the animals were pair-matched into treatment and control groups. Each group contains 8-10 mice, each of which was ear tagged and followed throughout the experiment.

The administration of drugs or controls began the day the animals were pair-matched (Day 1). Pumps (Alzet® Model 2002) with a flow rate of 0.5 μl/hr were implanted s.c. between the shoulder blades of each mice. Mice were weighed and tumor measurements were obtained using calipers twice weekly, starting Day 1. These tumor measurements were converted to mg tumor weight by standard formula, (W²×L)/2. The experiment is terminated when the control group tumor size reached an average of about 1 gram. Upon termination, the mice were weighed, sacrificed and their tumors excised. The tumors were weighed and the mean tumor weight per group was calculated. The change in mean treated tumor weight/the change in mean control tumor weight×100 (dT/dC) is subtracted from 100% to give the tumor growth inhibition (TGI) for each group.

### Example X

### Cardiac Glycoside Compounds Inhibits HIF-1α Expression

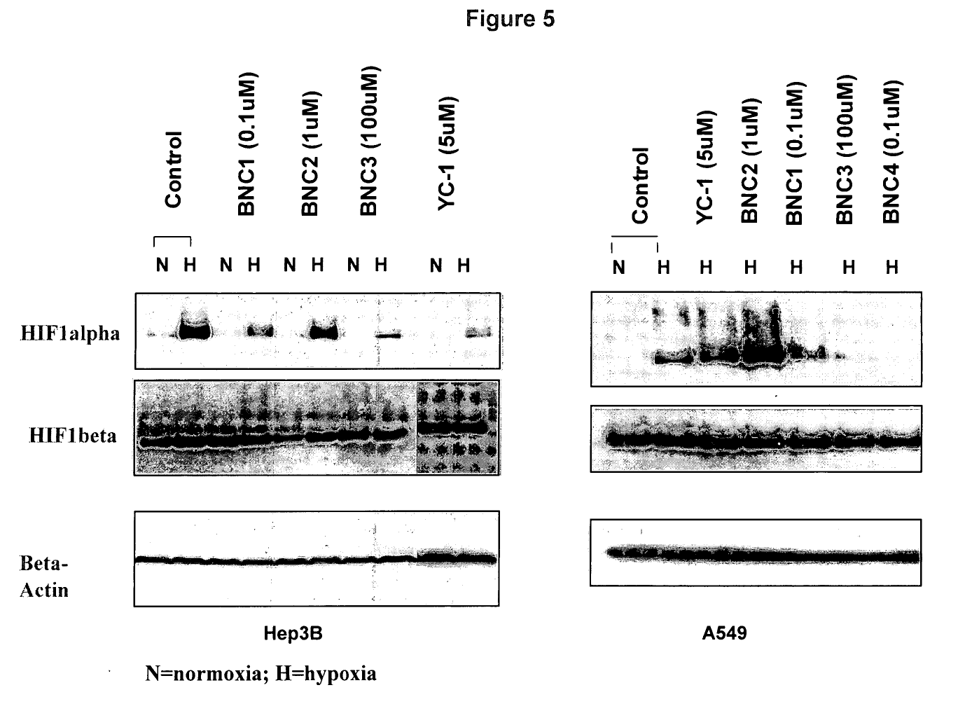

Cardiac glycoside compounds of the invention targets and inhibits the expression of HIF 1α based on Western Blot analysis using antibodies specific for HIF-1α (FIG. 5).

Hep3B or A549 cells were cultured in complete growth medium for 24 hours and treated for 4 hrs with the indicated cardiac glycoside compounds or controls under normoxia (N) or hypoxia (H) conditions. The cells were lysed and proteins were resolved by SDS-PAGE and transferred to a nylon membrane. The membrane was immunoblotted with anti-HIF-1α and anti-HIF1β MAb, and anti-beta-actin antibodies.

In Hep3B cells, various effective concentrations of BNC compounds (cardiac glycoside compounds of the invention) inhibits the expression of HIF-1α, but not HIF-1β. The basic observation is the same, with the exception of BNC2 at 1 μM concentration.

To study the mechanism of HIF-1α inhibition by the subject cardiac glycoside compounds, Hep3B cells were exposed to normoxia or hypoxia for 4 hrs in the presence or absence of: an antioxidant enzyme and reactive oxygen species (ROS) scavenger catalase (1000 U), prolyl-hydroxylase (PHD) inhibitor L-mimosine, or proteasome inhibitor MG132 as indicated. HIF-1α and β-actin protein level was determined by western blotting.

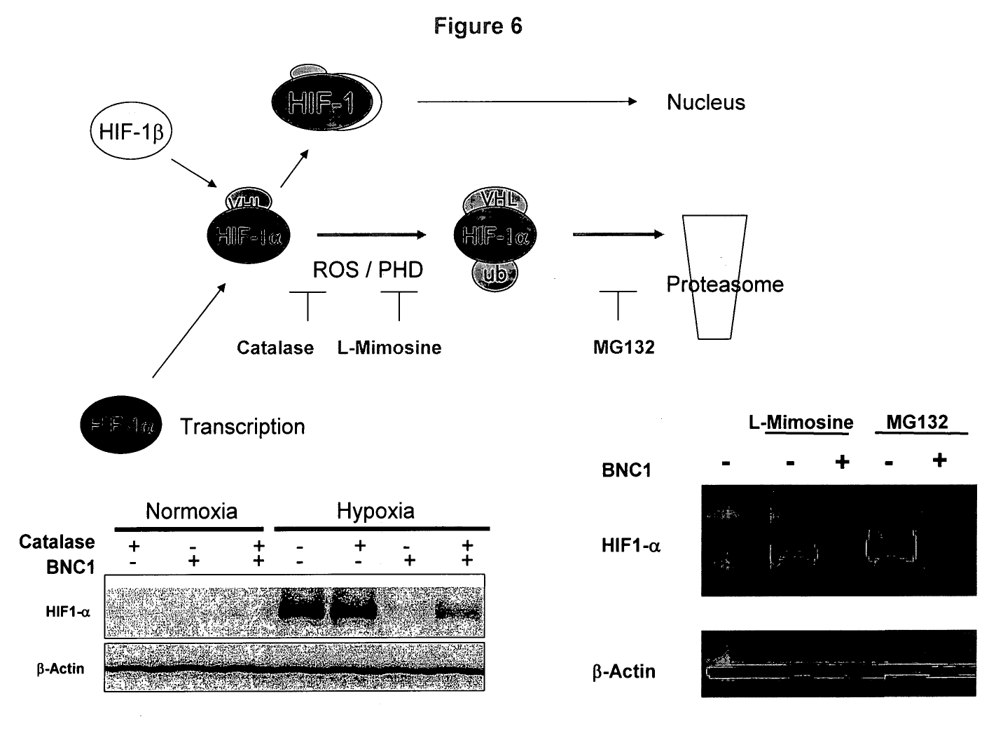

FIG. 6 indicates that the cardiac glycoside compound BNC-1 may inhibits steady state HIF-1α level through inhibiting the synthesis of HIF-1α.

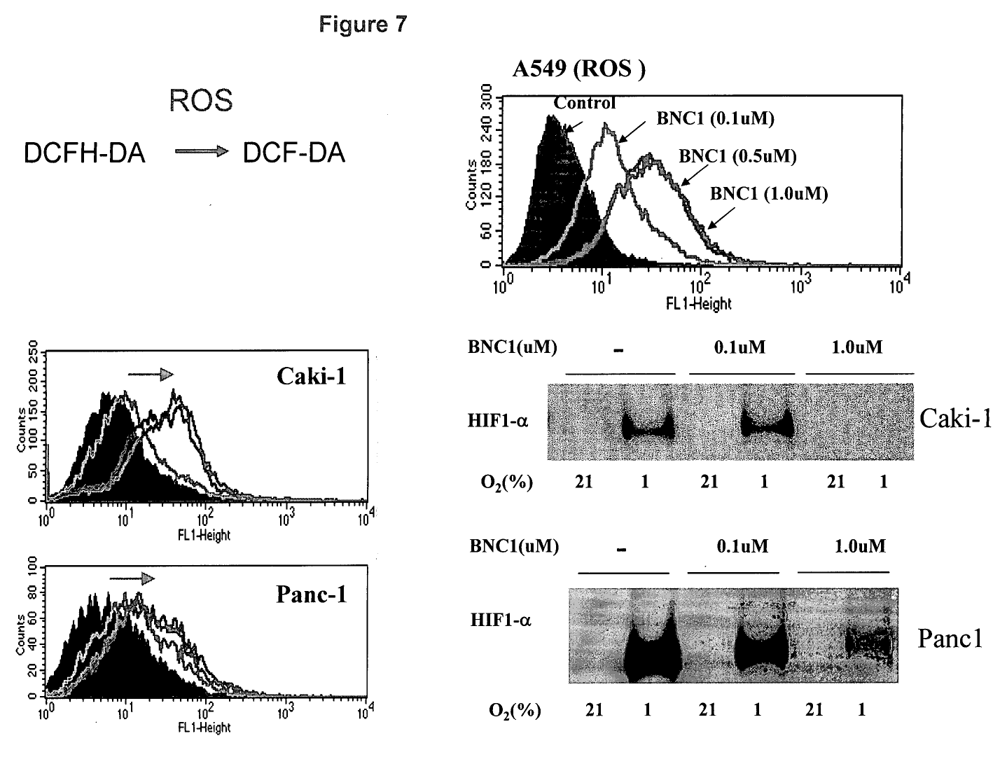

In a related study, tumor cell line A549(ROS) were incubated in normoxia in the absence (control) or presence of different amounts of BNC-1 for 4 hrs. Thirty minutes prior to the termination of incubation period, 2,7-dichlorofluorescin diacetate (CFH-DA, 10 mM) was added to the cells and incubated for the last 30 min at 37° C. The ROS levels were determined by FACS analysis. HIF-1α protein accumulation in Caki-1 and Panc-1 cells was determined by western blotting after incubating the cells for 4 hrs in normoxia (21% O₂) or hypoxia (1% O₂) in the presence or absence of BNC-1. FIG. 7 indicates that BNC-1 induces ROS production (at least as evidenced by the A549(ROS) Sentinel Lines), and inhibits HIF-1α protein accumulation in the test cells.

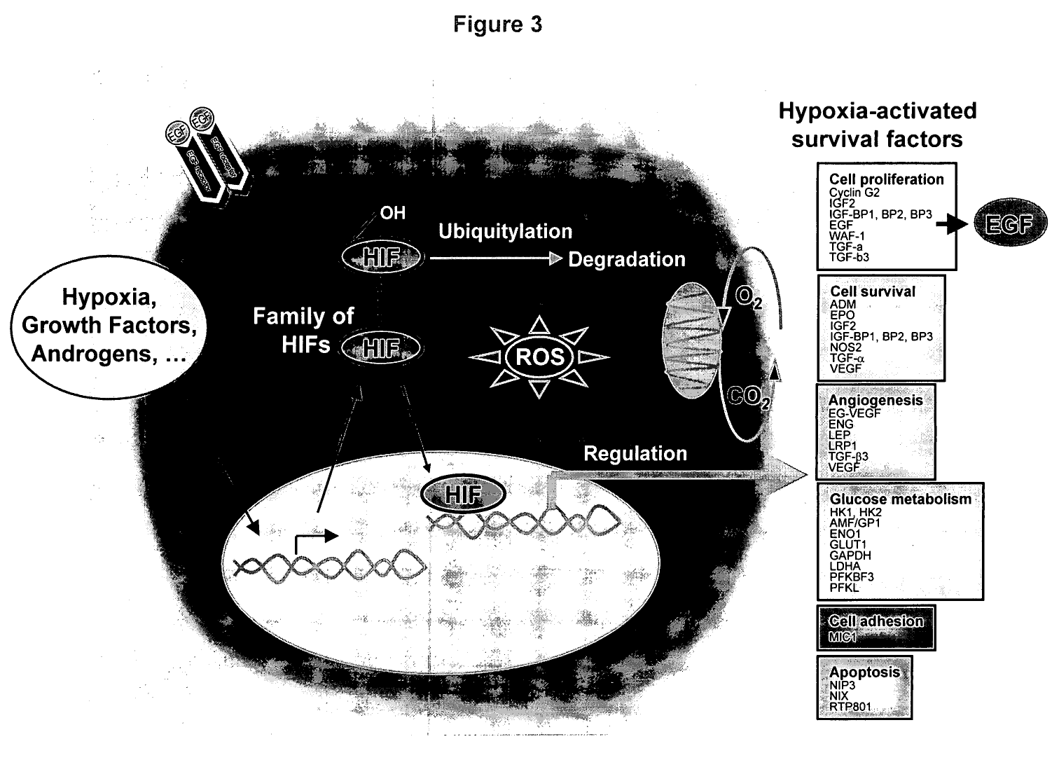

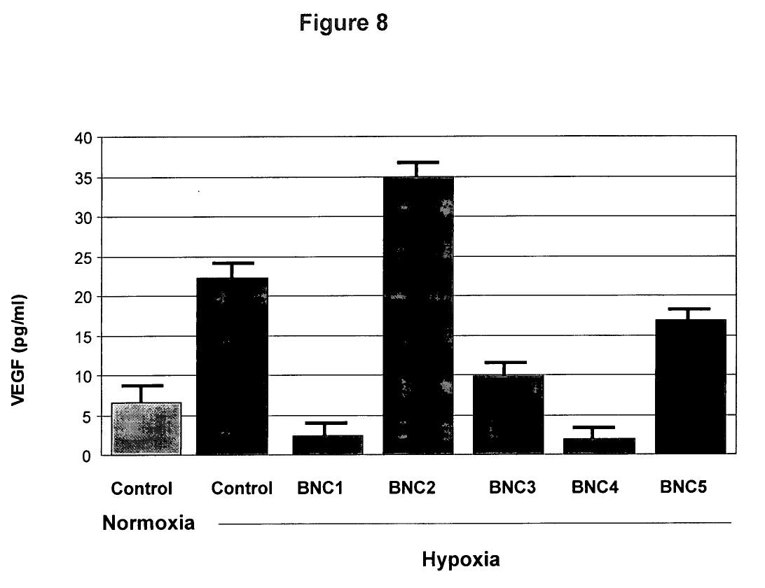

FIG. 8 also demonstrates that the cardiac glycoside compounds BNC-1 and BNC-4 directly or indirectly inhibits in tumor cells the secretion of the angiogenesis factor VEGF, which is a downstream effector of HIF-1α (see FIG. 3). In contrast, other non-cardiac glycoside compounds, BNC2, BNC3 and BNC5, do not inhibit, and in fact greatly enhances VEGF secretion.

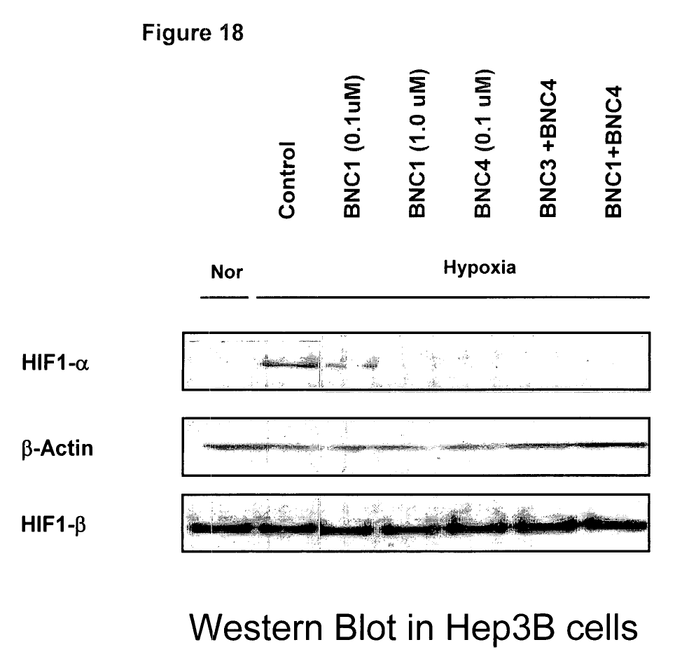

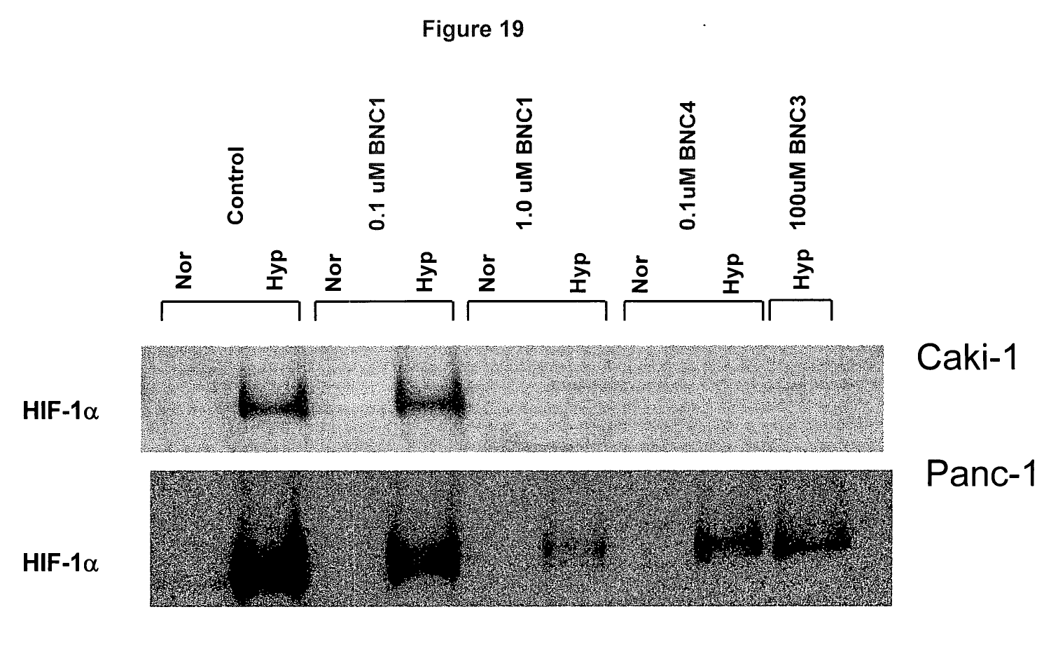

FIGS. 18 and 19 compared the ability of BNC-1 and BNC-4 in inhibiting hypoxia-mediated HIF-1α induction in human tumor cells. The figures show result of immunoblotting for HIF-1α, HIF-1β and β-actin (control) expression, in Hep3B, Caki-1 or Panc-1 cells treated with BNC-1 or BNC-4 under hypoxia. The results indicate that BNC-4 is even more potent (about 10-times more potent) than BNC-1 in inhibiting HIF-1α expression.

Thus, while not wishing to be bound by any particular theory, the ability of the subject compounds to treat refractory cancer may be at least partially related to their ability to inhibit HIF-1 α expression.

### Example XI

### Neutralization of Gemcitabine-induced Stress Response as Measured in A549 Sentinel Line

The cardiac glycoside compounds of the invention were found to be able to neutralize Gemcitabine-induced stress response in tumor cells, as measured in A549 Sentinel Lines.

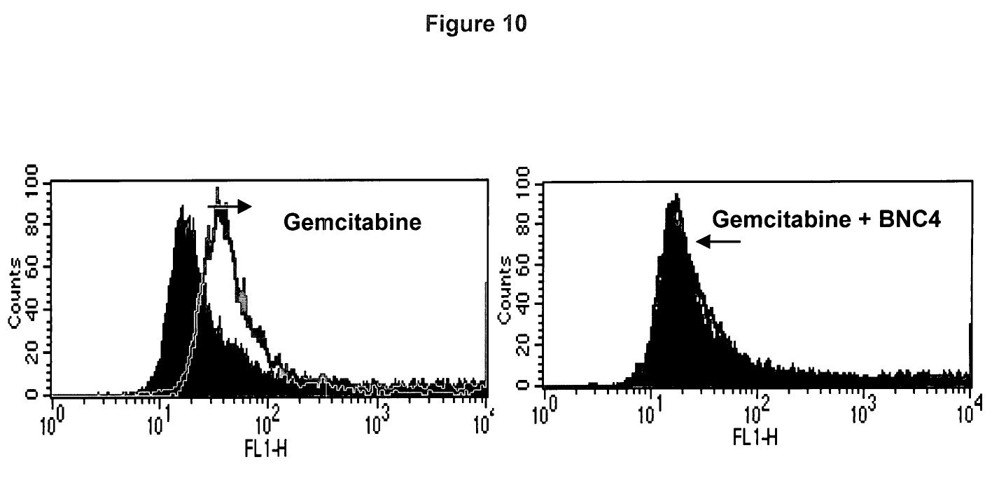

In experiments of FIG. 10, the A549 sentinel line was incubated with Gemcitabine in the presence or absence of indicated Bionaut compounds (including the cardiac glycoside compound BNC-4) for 40 hrs. The reporter activity was measured by FACS analysis.

It is evident that at least BNC-4 can significantly shift the reporter activity to the left, such that Gemcitabine and BNC-4-treated cells had the same reporter activity as that of the control cells. In contrast, cells treated with only Gemcitabine showed elevated reporter activity.

### Example XII

### Effect of BNC-1 Alone or in Combination with Standard Chemotherapy on Growth of Xenografted Human Pancreatic Tumors in Nude Mice

To test the ability of BNC-1 to inhibit xenographic tumor growth in nude mice, either along or in combination with a standard chemotherapeutic agent, such as Gemcitabine, Panc-1 tumors were injected subcutaneously (sc) into the flanks of male nude mice. After the tumors reached 80 mg in size, osmotic pumps (model 2002, Alzet Inc., flow rate 0.5 μl/hr) containing 20 mg/ml of BNC-1 were implanted sc on the opposite sides of the mice. The control animals received pumps containing vehicle (50% DMSO in DMEM). The mice treated with standard chemotherapy agent received intra-peritoneal injections of Gemcitabine at 40 mg/kg every 3 days for 4 treatments (q3d×4). Each data point represent average tumor weight (n=8) and error bars indicate SEM.

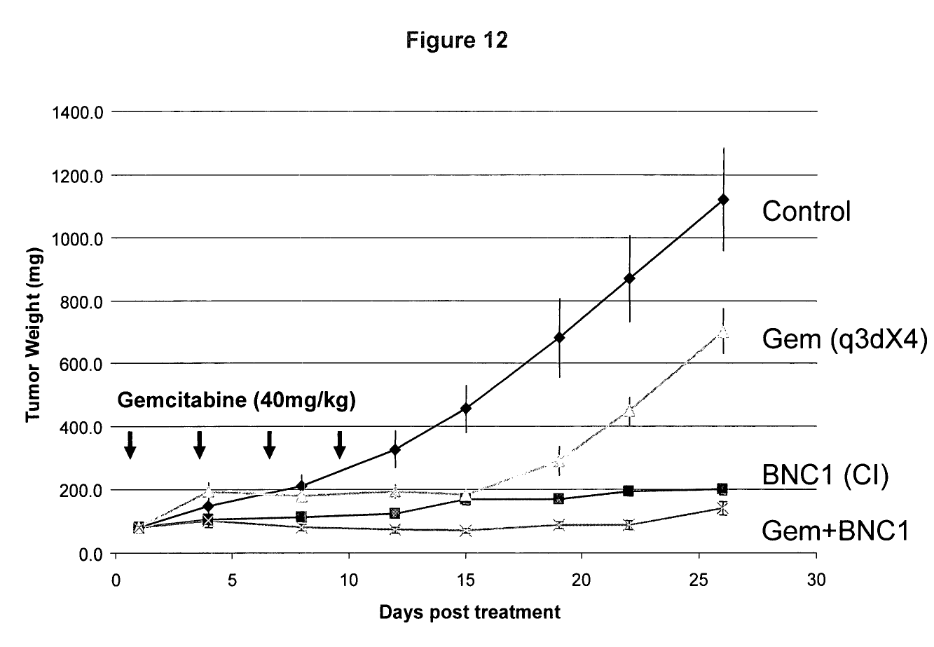

FIG. 12 indicates that, at the dosage tested, BNC-1 alone can significantly reduce tumor growth in this model. This anti-tumor activity is additive when BNC-1 is co-administered with a standard chemotherapeutic agent Gemcitabine. Results of the experiment is listed below:

| | | Final | | |
|---|---|---|---|---|
| Group | | Tumor weight | | |
| (Animal No.) | Dose/Route | Day 25 (Mean) | SEM | % TGI |
| Control (8) | Vehicle/i.v. | 1120.2 | 161.7 | — |
| BNC-1 (8) | 20 mg/ml; s.c.; C.I. | 200 | 17.9 | 82.15 |
| Gemcitabine (8) | 40 mg/kg; q3d × 4 | 701.3 | 72.9 | 37.40 |
| BNC-1 + Gem | Combine both | 140.8 | 21.1 | 87.43 |
| (8) | | | | |

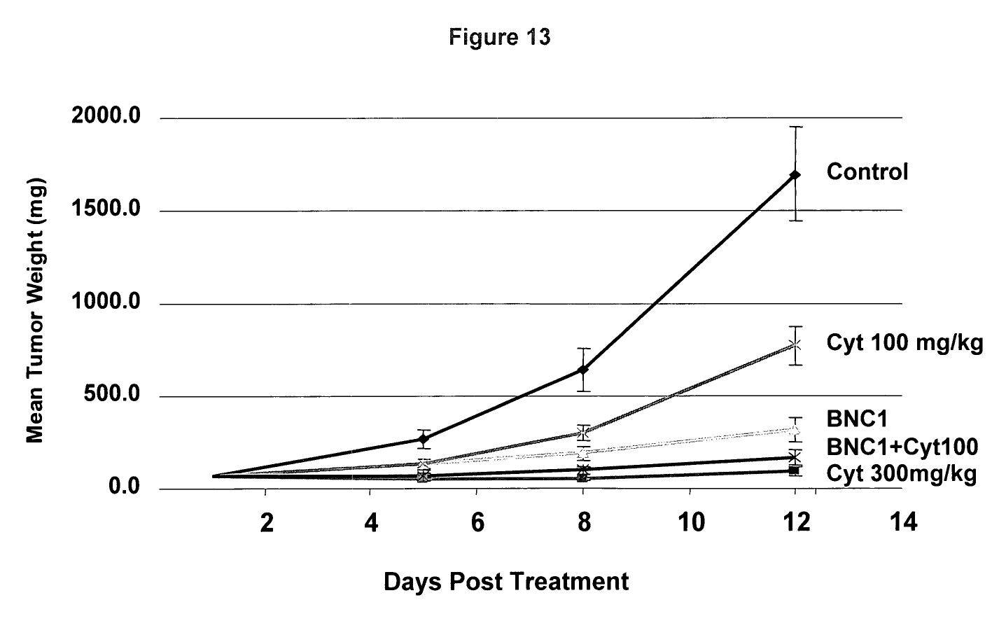

Similarly, in the experiment of FIG. 13, BNC-1 (20 mg/ml) was delivered by sc osmotic pumps (model 2002, Alzet Inc.) at 0.5 μl/hr throughout the study. Cytoxan (qld×1) was injected at 100 mg/kg (Cyt 100) or 300 mg/kg (Cyt 300). The results again shows that BNC-1 is a potent anti-tumor agent when used alone, and its effect is additive with other chemotherapeutic agents such as Cytoxan. The result of this study is listed in the table below:

| | | Final | | |
|---|---|---|---|---|
| Group | | Tumor weight | | % |
| (Animal No.) | Dose/Route | Day 22 (Mean) | SEM | TGI |
| Control (10) | Vehicle/i.v. | 1697.6 | 255.8 | — |
| BNC-1 (10) | 20 mg/ml; s.c.; C.I. | 314.9 | 67.6 | 81.45 |
| Cytoxan300 (10) | 300 mg/ml; ip; qd × 1 | 93.7 | 24.2 | 94.48 |
| Cytoxan100 (10) | 100 mg/ml; ip; qd × 2 | 769 | 103.2 | 54.70 |
| BNC-1 + | Combine both | 167 | 39.2 | 90.16 |
| Cytoxan100 (10) | | | | |

In yet another experiment, the anti-tumor activity of BNC-1 alone or in combination with Carboplatin was tested in A549 human non-small-cell-lung carcinoma. In this experiment, BNC-1 (20 mg/ml) was delivered by sc osmotic pumps (model 2002, Alzet Inc.) at 0.5 μl/hr throughout the study. Carboplatin (qld×1) was injected at 100 mg/kg (Carb).

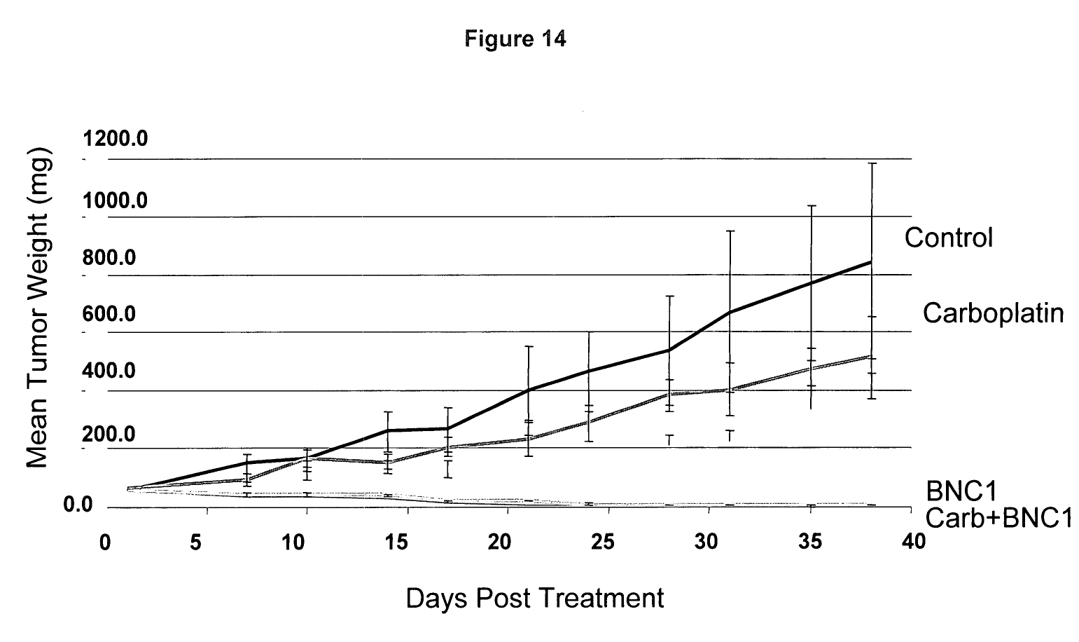

FIG. 14 confirms that either BNC-1 alone or in combination with Carboplatin has potent anti-tumor activity in this tumor model. The detailed results of the experiment is listed in the table below:

| | | % Weight | Final Tumor | | |
|---|---|---|---|---|---|
| Group | | Change at | weight Day 38 | | |
| (Animal No.) | Dose/Route | Day 38 | (Mean) | SEM | % TGI |
| Control (8) | Vehicle/i.v. | 21% | 842.6 | 278.1 | — |
| BNC-1 (8) | 20 mg/ml; s.c.; C.I. | 21% | 0.0 | 0.0 | 100.00 |
| Carboplatin (8) | 100 mg/kg; ip; qd × 1 | 16% | 509.75 | 90.3 | 39.50 |
| BNC-1 + Carb (8) | Combine both | 0% | 0.0 | 0.0 | 100.00 |

Notably, in both the BNC-1 and BNC-1/Carb treatment group, none of the experimental animals showed any signs of tumor at the end of the experiment, while all 8 experimental animals in control and Carb only treatment groups developed tumors of significant sizes.

Thus the cardiac glycoside compounds of the invention (e.g. BNC-1) either alone or in combination with many commonly used chemotherapeutic agents (e.g. Carboplatin, Gem, Cytoxan, etc.) has potent anti-tumor activities in various xenographic animal models of pancreatic cancer, renal cancer, hepatic, and non-small cell lung carcinoma.

### Example XIII

### Effect of BNC-4 Alone or in Combination with Standard Chemotherapy on Growth of Xenografted Tumors in Nude Mice

To test the ability of BNC-4 to inhibit xenographic tumor growth in nude mice, either along or in combination with a standard chemotherapeutic agent, such as Gemcitabine, Panc-1 tumors were injected subcutaneously (s.c.) into the flanks of male nude mice. After the tumors reached 80 mg in size, osmotic pumps (model 2002, Alzet Inc., flow rate 0.5 μl/hr) containing 15 mg/ml of BNC-4 were implanted sc on the opposite sides of the mice. The control animals received pumps containing vehicle (50% DMSO in DMEM). The mice treated with standard chemotherapy agent received intra-peritoneal injections of Gemcitabine at 40 mg/kg every 3 days for 4 treatments (q3d×4). Each data point represent average tumor weight (n=8) and error bars indicate SEM.

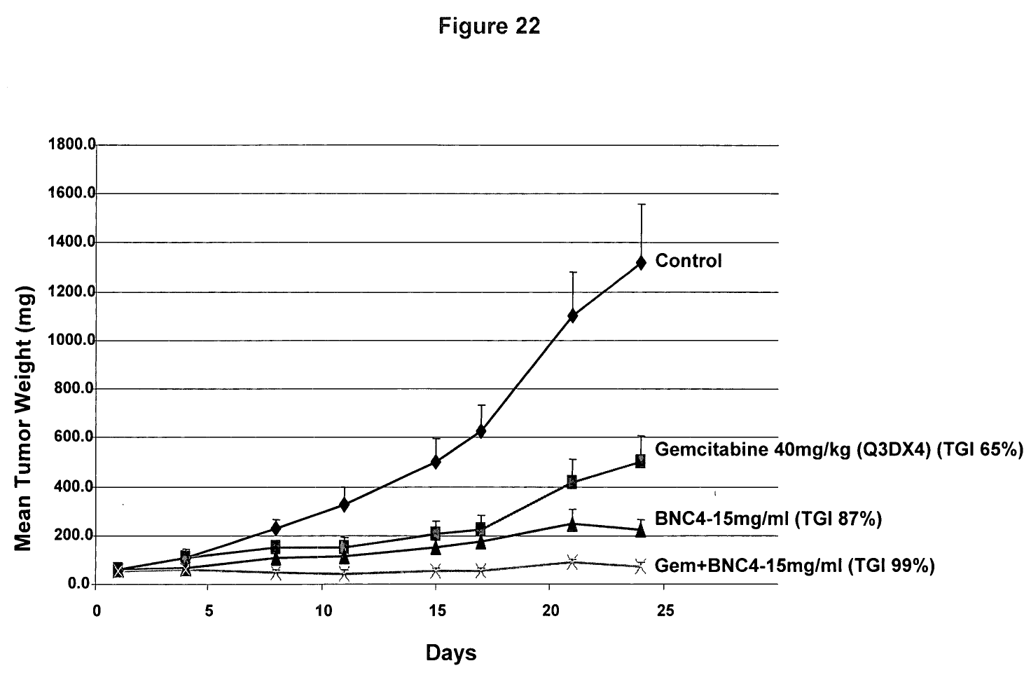

FIG. 22 indicates that, at the dosage tested, BNC-4 alone can significantly reduce tumor growth in this model. The TGI is about 87%, compared to 65% of the Gemcitabine treatment. This anti-tumor activity is additive when BNC-4 is co-administered with a standard chemotherapeutic agent Gemcitabine, with a TGI of about 99%.

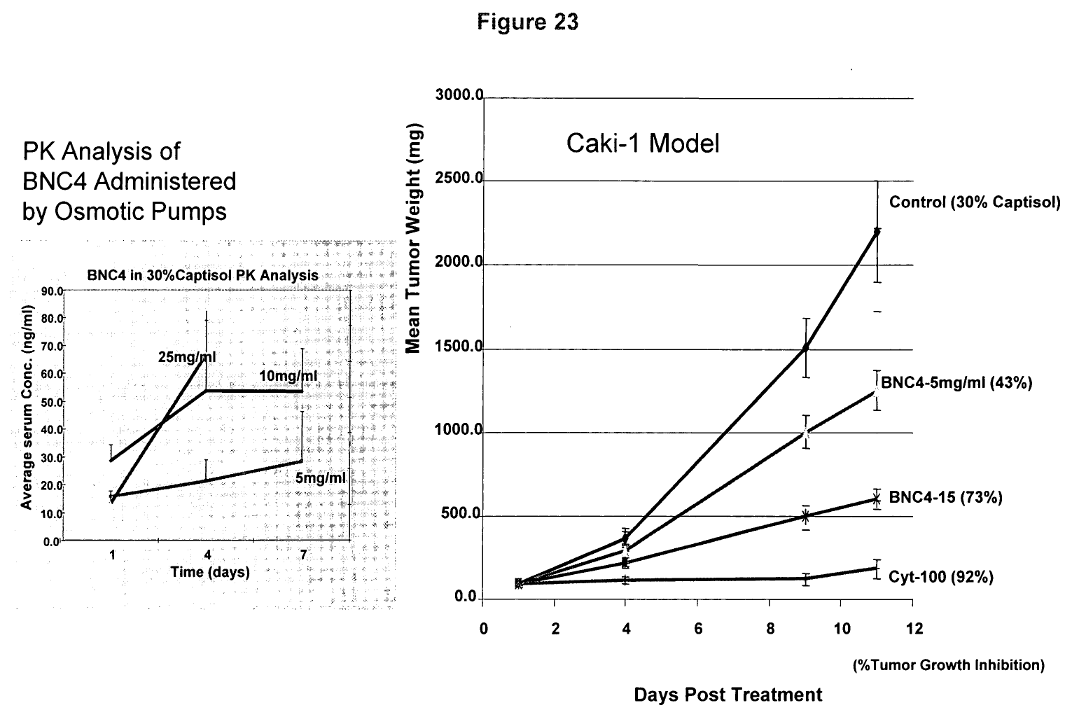

Similarly, in the experiment of FIG. 23, where renal cancer cell line Caki-1 was injected into nude mice, BNC-4 (5 or 15 mg/ml) was delivered by sc osmotic pumps (model 2002, Alzet Inc.) at 0.5 μl/hr throughout the study. Cytoxan (qld×1) was injected at 100 mg/kg (Cyt 100). The results again showed that BNC-4 is a potent anti-tumor agent when used alone (TGI of 73% and 43% for the 15 and 5 mg/ml treatment groups, respectively). As a positive control, Cytoxan achieved a 92% TGI when used alone.

Thus the cardiac glycoside compounds of the invention (e.g. BNC-4) either alone or in combination with many commonly used chemotherapeutic agents (e.g. Gem, Cytoxan, etc.) has potent anti-tumor activities in various xenographic animal models, including pancreatic cancer and renal cancer.

Pharmacokinetic studies of the BNC-4 delivered by osmotic pump were also conducted. The results of average serum concentrations of BNC-4, over the course of 1-7 days, were plotted in the left panel of FIG. 23.

### Example XIV

### Determining Minimum Effective Dose

Given the additive effect of the subject cardiac glycosides with the traditional chemotherapeutic agents, it is desirable to explore the minimal effective doses of the subject cardiac glycosides for use in conjoint therapy with the other anti-tumor agents.

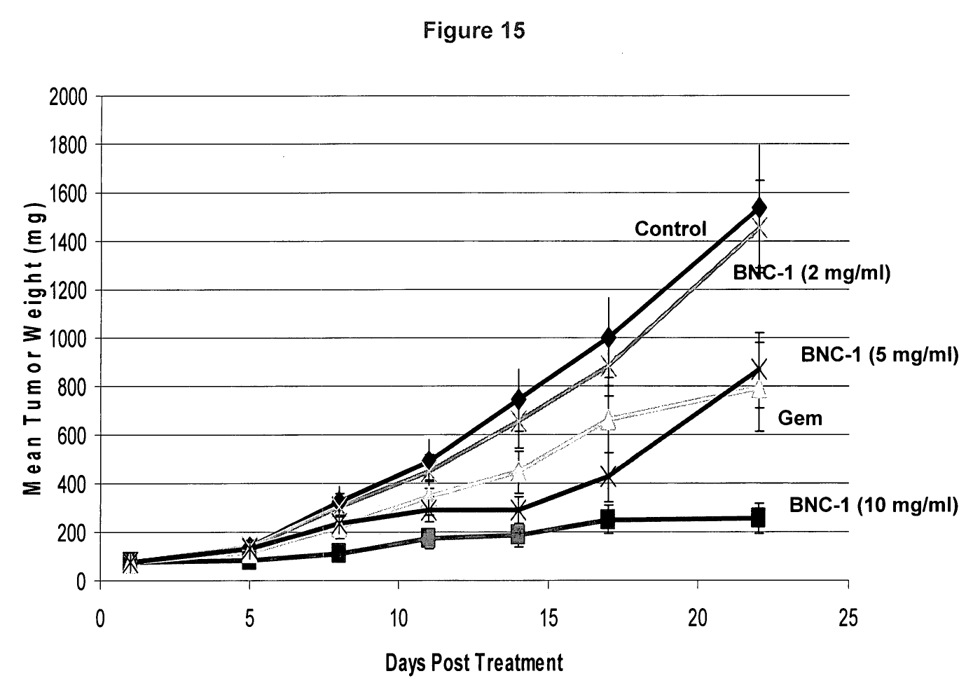

FIG. 15 shows the titration of the exemplary cardiac glycoside BNC-1 to determine its minimum effective dose, effective against Panc-1 human pancreatic xenograft in nude mice. BNC-1 (sc, osmotic pumps) was first tested at 10, 5 and 2 mg/ml. Gem was also included in the experiment as a comparison.

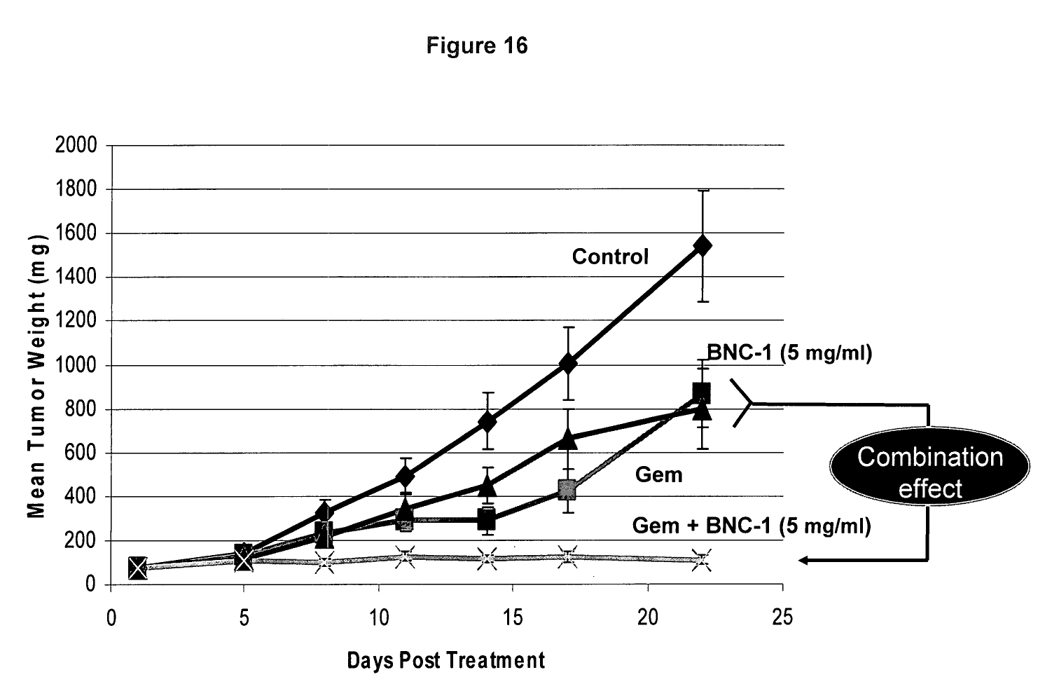

FIG. 16 shows that combination therapy using both Gem and BNC-1 produces a combination effect, such that sub-optimal doses of both Gem and BNC-1, when used together, produce the maximal effect only achieved by higher doses of individual agents alone.

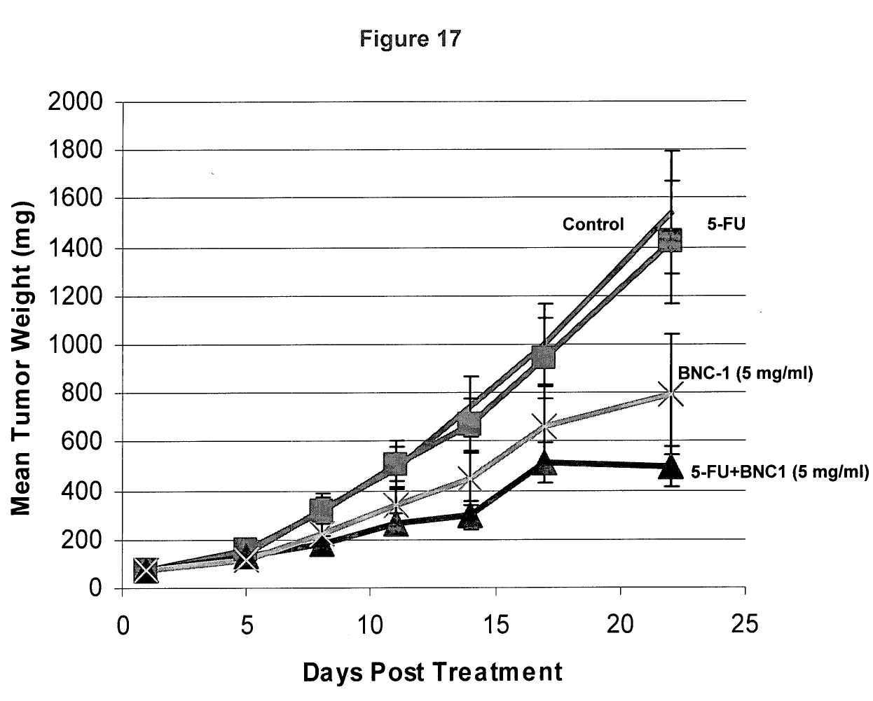

A similar experiment was conducted using BNC-1 and 5-FU, and the same combination effect was seen (see FIG. 17).

Similar results are also obtained for other compounds (e.g. BNC2) that are not cardiac glycoside compounds (data not shown).

### Example XV

### BNC-1 and BNC-4 Inhibit HIF-1α Induced Under Normoxia by PHD Inhibitor

As an attempt to study the mechanism of BNC-4 inhibition of HIF-1α, we tested the ability of BNC-1 and BNC-4 to inhibit HIF-1α expression induced by a PHD inhibitor, L-mimosone (see FIG. 6), under normoxia condition.

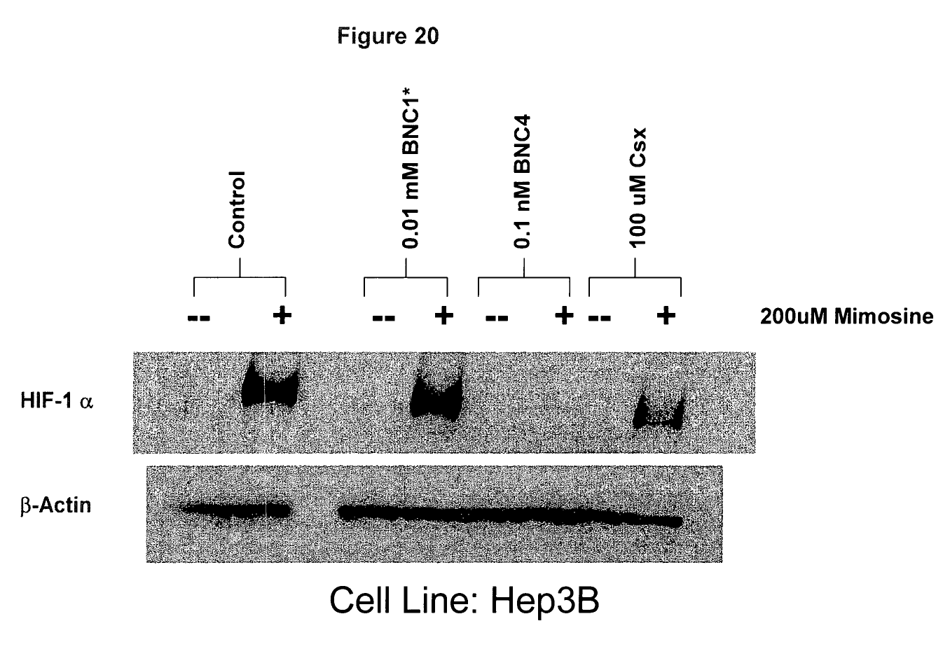

In the experiment represented in FIG. 20, Hep3B cells were grown under normoxia, but were also treated as indicated with 200 μM L-mimosone for 18 h in the presence or absence of BNC-1 or BNC-4. Abundance of HIF-1α and β-actin was determined by western blotting.

The results indicate that L-mimosone induced HIF-1α accumulation under normoxia condition, and addition of BNC-4 or BNC-1 eliminated HIF-1α accumulation by L-mimosone. At the low concentration tested, BNC-1 and BNC-4 did not appear to have an effect on HIF-1α accumulation in this experiment. While not wishing to be bound by any particular theory, the fact that BNC-4 and BNC-1 can inhibit HIF-1α induced under normoxia by PHD inhibitor indicates that the site of action by BNC-4 probably lies up-stream of prolyl-hydroxylation.

### Example XVI

### BNC-4 Inhibits Na/K-ATPase Activity and has Anti-HIF/Anti-Proliferative Activity

To determine whether there is a correlation and hence validate that the observed anti-HIF/anti-Proliferative activity effects are due to an on target inhibition of Na⁺/K⁺-ATPase activity by BNC-4 and its related compounds, we measured the inhibition of Na⁺/K⁺-ATPase by BNC-4, its closely related compound BNC-151, and the aglycone BNC-147.

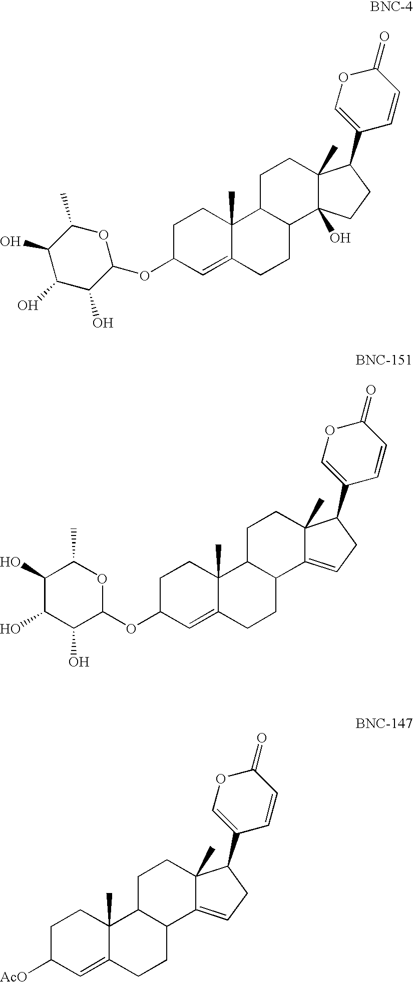

The results indicates that BNC-4 is about 10-times more potent than BNC-151, with an IC50 of about 130 nM (compared to 1380 nM for BNC-151 and 65,000 nM for BNC-147).

BNC-4 is even more potent in inhibiting cancer cell proliferation. In an anti-proliferation assay measuring % MTS activity in the A549 cell line, the IC50 for BNC-4 is only about 2.1 nM (compared to that of 260 nM for BNC-151, and 11500 nM for BNC-147).

Western blot using anti-HIF-1α antibody showed that BNC-4 completely inhibits HIF-1α expression at both 1 μM and 0.1 μM. Significant inhibition of HIF-1α expression was also observed for BNC-151 at 1 μM, and 0.1 μM to a lesser extent.

### Example XVII

### The Bufadienolides are More Potent in Activity than the Cardenolides

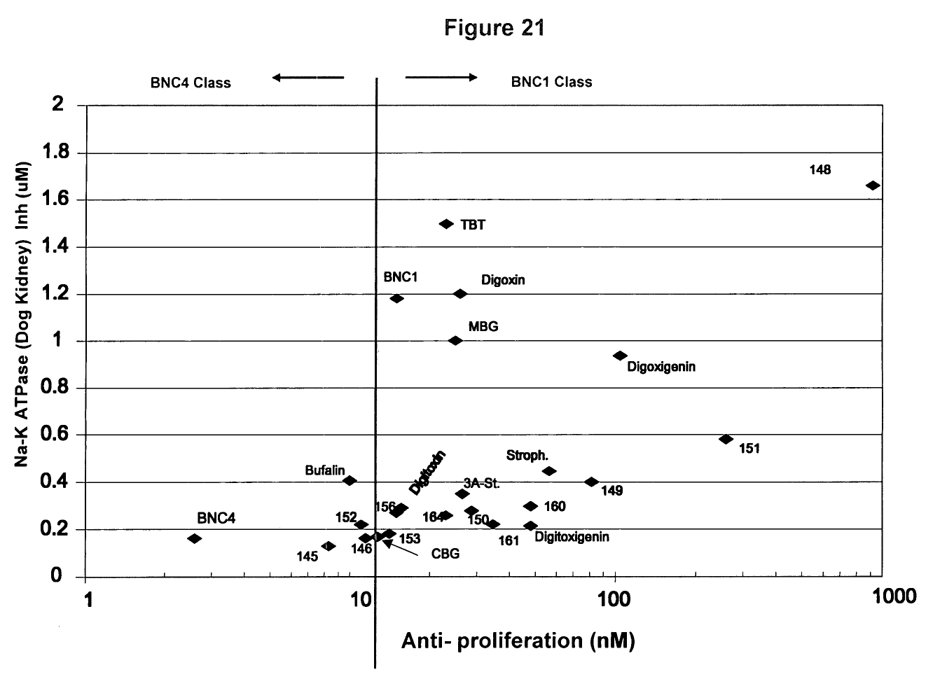

To validate correlation between Na⁺/K⁺-ATPase activity and identify best in class, in terms of anti-prolferative activity we conducted experiments to profile various known cardiac glycosides and different analogues of BNC-4 for their anti-prolerafitive and anti-Na⁺/K⁺-ATPase activity. The relative activity of the bufadienolide class of cardiac glycosides was determined to be much greater then cardenolide class, Anti-prolerafitive IC₅₀ values were determined by MTS assay using an A549 cell line. Na⁺/K⁺-ATPase inhibition IC₅₀ values were obtained using enzyme preparation from dog kidney (Sigma). The results of these assays were summarized in FIG. 21.

It is apparent that the a correlation between Na⁺/K⁺-ATPase activity and anti-proleferative activity is present and that the bufadienolides are generally more potent than the cardenolides as Na⁺/K⁺-ATPase inhibitors and anti-proliferation agents.

The subject bufadienolides and aglycones thereof preferably have anti-proliferation IC₅₀ of less than about 500 nM, more preferably less than about 11 nM, 10 nM, 5 nM, 4, nM, 3 nM, 2 nM, or 1 nM.

The subject bufadienolides and aglycones thereof preferably have anti-Na/K-ATPase IC₅₀ of less than about 0.4 μM, more preferably less than about 0.3 μM, 0.2 μM, or 0.1 μM.

In contrast, the subject cardenolides generally have anti-proliferation IC₅₀ of about 10-500 nM (see FIG. 21).

Experiments were also conducted to demonstrate that there is an inverse correlation between target Na⁺/K⁺-ATPase levels in cancer cell lines, and the anti-proliferative activity of the cardiac glycosides (e.g., bufadienolides, such as BNC-4).

Specifically, the anti-proliferative IC₅₀ values were determined for 11 established cell lines from various cancers, namely A549, PC-3, CCRF-CEM, 786-0, MCF-7, HT-29, Hop 18, SNB78, IGR-OV1, SNB75, and RPMI-8226. These cancer cell lines have different amounts of isoform-1 and isoform-3 of Na/K-ATPase, and the total amount of the two isoforms in each cell line were determined by quantitating the mRNA levels of the two isoforms by real time RT-PCR (TaqMan), using fluorescent labeled TaqMan probes. The anti-proliferation IC₅₀ values were determined by MTS assay as above. The results were plotted (total level of target Na/K-ATPase mRNA v. IC₅₀).

The measured IC₅₀ values range between 3.5-18.2 nM, while the total mRNA levels varied between 261-1321 arbitrary units. And the correlation coefficient (R) value was determined to be −0.73.

### Example XVIII

### Dosage Forms and Pharmacokinetic Studies for BNC-4/Proscillaridin

This example provides a typical pharmacokinetic study for one exemplary bufadienolide cardiac glycosides-proscillaridin. Similar studies may be carried out for any of the other cardiac glycosides that can be used in the instant invention.

A. Therapeutic Use and Approval Status:

Proscillaridin was first introduced in Germany in 1964 by Knoll AG (now Abbott) (Talusin®), by Sandoz (now Novartis) (Sandoscill®), and other companies as an alternative to Ouabain (g-Strophanthin) and Digoxin/Digitoxin for acute and chronic therapy of congestive heart failure. Since then the substance was approved in Australia, Austria, Finland, France, Greece, Italy, Japan, the Netherlands, New Zealand, Norway, Poland, Portugal, Russia (and other countries of the former Soviet Block), Spain, Sweden, Switzerland, and several countries in South America (e.g. Brazil, Argentina). However, Proscillaridin has never been approved for any indications in the U.S. Trade names include Caradrin, Cardimarin, Cardion, Encordin, Neo Gratusimal, Procardin, Proscillaridin, Prosiladin, Protosin, Proszin, Sandoscill, Scillaridin, Scillarist, Stellarid, Talusin, Theocaradrin, Theostellarid, Theotalusin, Tradenal, Tromscillan, etc. Thus “Proscillaridin” as used herein includes all forms of these compounds and their minor variants.

Numerous scientific papers have been published in the literature on the chemistry, pharmacology, uses and usefulness of Proscillaridin and related compounds. However, with the advent of ACE-inhibitors and latergeneration beta-blockers, the therapeutic use of cardiac glycosides has been on the retreat, only Digoxin being still widely prescribed.

B. Cardiac Pharmacology:

Basically, Proscillaridin shares its cardio active action with other cardiac glycosides such as Digoxin or Ouabain. The contraction of the myocardium is increased (positive inotropic effect), frequency and electric stimulus transduction are decreased (negative chronotropic effect); at low doses the transduction threshold is decreased, while it increases at higher doses. The latter effect can lead to heterotopic stimuli such as extra-systoles and arrhythmia, which are part of the pattern of symptoms appearing at intoxication levels.

The molecular mechanism of the cardiac action of Proscillaridin is more-or-less identical to that of the other cardiac glycosides, and centers on the modulation/inhibition of the sarcoplasmic Na/K-ATPase ion pump. This trans-membrane protein exchanges three cytosolic sodium ions for two extra-cellular potassium ions at the expense of ATP. The Na/K-ATPase protein consists of two subunits (α and β), which are assembled on demand together with a third (γ) subunit to form the functional enzyme complex. The α- and β-subunits come in different isoforms (so far 4 isoforms have been described for the α-subunit, and 3 for the β-subunit), which allows for a large variety of Na/K-ATPase isoforms to exist. The different variations are tissue-specific, and show different affinities towards cardiac glycosides. This explains the specific high sensitivity of myocardial muscle fibers and adrenergic nerve cell membranes towards cardiac glycosides.

For example, based on Western blot analysis, the alpha1 isoform of Na/K-ATPase is constitutively expressed in most organisms tested, including brain, heart, smooth intestine, kidney, liver, lung, skeletal muscle, testis, spleen, pancrease, and ovary, with the most abundant expression observed in brain and kidney. The alpha2 isoform is largely expressed in the brain, muscle, and heart. The alpha3 isoform is rich in the CNS, especially the brain. The alpha4 isoform appears to be specific for the testis.

There exist two binding sites for cardiac glycosides among the Na/K-ATPase α-subunits: a high-affinity/low-density site, and a low-affinity/high-density site. About 25% of all binding sites on ventricular muscle cells are of the high affinity type (Akera T et al. 1986). Very small amounts of cardiac glycosides (e.g., Ouabain) stimulate rather than inhibit sodium pump action, presumably by interacting with the high-affinity binding sites (Gao et al. 2002). These binding events trigger a variety of signal cascades involved in cellular growth by controlling the binding of the α-subunit to Caveolin-1, an essential protein for intra-cellular signal-transduction and vesicular trafficking (Wang H et al. 2004). At higher local concentrations of cardiac glycoside also the low-affinity binding site becomes involved, and the overall enzyme exchange rate diminishes. This results in a net loss of intracellular potassium, leading to a sodium imbalance, which is in turn offset by calcium influx by way of the Na/Ca-exchanger. The increased concentration of intracellular calcium leads to a higher contractility of the myocardial cells, resulting in a stronger and more complete contraction of the heart muscle.

In a comparative study of therapeutically used cardiac glycosides the order of Na/K-ATPase-inhibition was Ouabain<Digoxin<Proscillaridin, making Proscillaridin one of the most potent modulators of the sodium pump (Erdmann E 1978). (For a comprehensive overview on the molecular- and clinical pharmacology of Cardiac Glycosides in general, and Digitalis Glycosides in particular, see: Karl Greeff (Ed.) “Cardiac Glycosides”, 2 Vols., Springer Verlag, 1981; and: Thomas Woodward Smith (Ed.) “Digitalis Glycosides”, Grune & Stratton 1986).

C. Anti-Cancer Indication and Mechanism-of-Action:

Proscillaridin A is a potent cytotoxic agent against a panel of 10 cancer cell lines, with a median IC₅₀ of about 23 nM (compared with 37 nM for Digoxin, and 78 nM for Ouabain).

While not wishing to be bound by any particular theory, the theory that cardiac glycosides, such as Proscillaridin, exerts their effect through acting on the sodium pump (Na/K-ATPase) is an attractive model for explaining the anti-cancer activity of cardiac glycosides in general and Proscillaridin in particular.

On one hand, there is ample evidence that increased intracellular calcium concentrations disturb the action potential across the mitochondrial membrane, increasing the uncontrolled proliferation of reactive oxygen species (ROS) and triggering apoptotic cascades. On the other hand, glycoside binding to the Na/K-ATPase is by itself a signaling event, inducing the Src-EGFr-ERK pathway, activating protein tyrosine phosphorylation and mitogen-activated protein kinases (MAPK), and increasing the production of ROS (see, for example: Tian J, Gong X, Xie Z. 2001. Ferrandi M et al. 2004).

Applicants have found for the first time that Ouabain and, to an even larger degree, BNC-4 (Proscillaridin) induce a signal that prevents cancer cells to respond to hypoxic stress through transcriptional inhibition of Hypoxia Inducible Factor (HIF-1α) biosynthesis. This may form the basis of the observed anti-cancer activity of cardiac glycosides, such as Proscillaridin, and their aglycones.

While not wishing to be bound by any particular theory, cancer cells of solid tumors are poorly vascularized, and, as a consequence, permanently exposed to sub-normal oxygen levels. As a response, they over-produce HIF. HIF1-α functions as an intracellular sensor for hypoxia and the presence of ROS. In normoxic cells, HIF1-α is continuously degraded by oxidative hydroxylation involving the enzyme proline-hydroxylase. Lack of oxygen prevents this degradation, and allows HIF to be transformed into a potent nuclear transcription factor. Its multi-valency makes it a central turn-on switch for the transcription of a wide variety of growth factors and angiogenic factors that are essential for malignant survival, growth and metastasis. By inhibiting HIF1-α biosynthesis, BNC-4 prevents cancer cells from producing these factors, and hence from proliferating, invasion, and metastasis.

Since in cancer cells, the distribution and combination of isoforms of the sodium pump, and hence the sensitivity towards cardiac glycosides is often dramatically altered, treatment with BNC-4 and its analogs allow cancer-specific molecular intervention with minimum effects on healthy tissues (Sakai et al. 2004, and references cited therein).

D. Pharmacokinetics:

a) Absorption:

Orally dosed Proscillaridin is rapidly, yet incompletely absorbed. The reported values range from 7 to 40%, with an accepted median at about 20%. These values were determined, however, with simple oral formulations (hydroalcoholic solutions or tablets), comparing i.v. and oral doses necessary to achieve pulse normalization in tachycardic patients (Hansel 1968; Belz 1968).

It has become evident that exposing Proscillaridin to stomach acid causes substantial decomposition (Andersson K E et al. 1976, 1975b; Einig H 1976). Thus the invention provides special dosage forms for certain patients, such as those taking antacids routinely, because in these patients, there is decreased stomach acid production, resulting in up to 60% higher absorption of Proscillaridin (Andersson K E 1977c). Proper adjustments are made in these special dosage forms to ensure the same final serum concentration effective for cancer treatment.

In other embodiments, the subject oral formulations mitigates this acid instability by including an acid-resistant coating, such as an enteric coating. With such a dosage form, absolute bioavailability is increased to about 35%. These data show that orally dosed Proscillaridin is being absorbed and distributed at a significant and measurable level, and behaves in this respect not differently from many other successful drugs with rapid first-pass metabolism (Pond S M, Toser T N 1984).

b) Distribution

After oral administration, peak blood concentrations of unconjugated Proscillaridin are reached after 15-30 minutes (Belz G G et al. 1973, 1974; Andersson K E et al. 1977a). However, the absolute value of measurable unconjugated drug reflects only 7% of the administered quantity, most likely a consequence of the formulation used in the experiment, the instability in gastric juices, and extensive first-pass metabolism (conjugation) in the gut wall (see below). The striking difference between portal and peripheral blood indicates a rapid tissue distribution.

Monitoring blood levels at 10-minutes intervals reveals a second, longer-lasting peak at about 1 hour: at this time, equilibrium between free and bound drug has been reached. Measuring of plasma concentrations over a longer period reveals that a third peak is reached at about 10 hours after dosing (Belz G G et al. 1974). This multi-phasic distribution is characteristic for entero-hepatic recycling of cardiac glycosides: the conjugates are excreted into the intestine, cleaved by the local bacteria, and the de-conjugated drug is re-absorbed (Andersson K E et al 1977b).

For clinical purposes it is important to know that optimal therapeutic plasma levels (EC) can be achieved with a single oral dose of 3.5 mg in as short as 30 minutes, and steady state is reached after 48 to 72 hours by continuing doses of 1.0 to 1.5 mg/d (Heierli C et al. 1971) (see “Posology” below). At this level about 85% of the substance is bound to plasma protein (Kobinger W, Wenzel W 1970).

Intravenous injection of 0.9 mg produced a plasma concentration of 1.09 ng/mL (measured by ⁸⁶Rb-uptake; Belz G G et al. 1974a), giving a Volume-of-Distribution (V_{D}) of 562 liters; this comparatively large value indicates an extensive tissue distribution typical for cardiac glycosides (compare to V_{D} for Digoxin-650 liters).

In this context, it is important to note the differences in measurable plasma drug levels depends on the method used. In contrast to the values obtained by ⁸⁶Rb-uptake, radio-immunoassays of plasma samples from 12 healthy individuals receiving 2×0.5 mg Talusin for 8 days gave a median Cₘₐₓ of 23.5±2.6 ng/mL, and Tₘₐₓ of 0.8±0.5 hours, with a median AUC of 385.0±43.6 ng/mL×h (Buehrens K G et al. 1991). While the former method measures only un-conjugated glycoside, which has to be extracted with dichloromethane prior to measurement, RAIs and ELISAs can be applied directly to plasma samples and measure free and conjugated drug together. Considering that the conjugates are still bioactive, the latter methods deliver probably a more indicative picture for the present indication. Unless specifically indicated otherwise, the serum concentration used herein refers to the total concentration of the subject cardiac glycosides, including conjugated/unconjugated forms bound or unbound by serum proteins.

c) Metabolism and Excretion:

For Proscillaridin, the total level of metabolism is >95%. In the stomach the glycosidic linkage is hydrolytically cleaved to a large extent, depending on the formulation used. Nevertheless, the de-glycosylated aglycone (e.g., Scillarenin for Proscillaridin and Scillaren) has a similar biological activity, and is also absorbed by the gut. During passage through the gut wall and subsequent liver passage, the substance becomes conjugated to glucuronic acid and sulfuric acid, and is secreted predominantly with bile. Subsequent de-conjugation by intestinal bacteria leads to partial re-absorption, resulting in the bi-phasic excretion profile mentioned above (Andersson K E et al. 1977b). Oxidative metabolism by P450 enzymes is much less pronounced, leading again to cleavage of the sugar linkage. Greater than 99% of the drug and its metabolites are excreted by the bile, while less than 1% of unchanged Proscillaridin is excreted by the kidneys. This independence of excretion from renal function makes the drug especially valuable for the treatment of patients with acute or chronic kidney disease, such as (refractory) renal cancer.

d) Plasma Concentration and Clearance:

The median plasma half-life (T_{1/2}) of Proscillaridin range from 23 to 29 hours in healthy individuals, and up to 49 hours median in cardiac patients (Belz G G, Brech W J 1974; Belz G G, Rudofsky G et al. 1974; Bergdahl B 1979), with daily clearance being ˜35%. The latter value is very different from those for Digitalis glycosides, which makes Proscillaridin the preferred drug when good control and quick dose adjustment to negative effects is essential.

Because the drug is almost entirely excreted through the bile, impaired kidney function has no influence on clearance (Belz G G, Brech W J 1974).

The measurements of therapeutic plasma levels at steady state vary, depending on the analytical methodology used (see above). Measuring the uptake of the Rubidium isotope ⁸⁶Rb by erythrocytes exposed to plasma gives values of circulating un-conjugated un-bound Proscillaridin ranging from 0.2 to 1.0 ng/mL (Cₘₐₓ) (Belz G G et al. 1974a); radio-immune assays on the other hand, do not distinguish between un-conjugated and conjugated or plasma-bound vs. free drug, and show levels between 10 and 30 ng/mL. It is probable that therapeutic action is also produced by the plasma-bound drug, and, albeit probably to a lesser extent, by the conjugates, as has been shown for Digoxin (Scholz H, Schmitz W 1984). Conjugate concentrations in blood plasma reached almost 20 ng/mL after a single oral dose of 1.5 mg Proscillaridin (Andersson et al 1977a).

Nevertheless, the median effective concentration (EC₅₀) of free Proscillaridin for cardiac indications is about 0.8 ng/mL (Belz G G et al. 1974c), which can be maintained by a median oral dosage of 0.9 mg/d (Loeschhorn N 1969). The median effective concentration (EC₅₀) of free Proscillaridin for the subject cancer indications is about 1.5 to 3 times that of cardiac indications, or about 1.2-2.5 ng/mL of free (unbound, unconjugated) Proscillaridin.

e) Posology:

In cardiac patients, at doses of 1.5 mg/d, steady states of therapeutic plasma levels are reached after 3 to 5 days (loading-to-saturation) with very few side-effects reported. The duration of cardiac action after saturation lies between 2 and 3 days. The optimal therapeutic level for cardio-vascular indications (ED_{p.o.}) was determined to be close to 5 mg by measuring the amount necessary to normalize tachycardia/fibrillation. Thus a one-time dose of 3.5 mg/d, followed by maintenance doses of 1.5 mg for two days and 1 mg/d thereafter can achieve this level in about 60 hours (Heierli C. et al. 1971; Hansel 1968). Belz determined the optimal median maintenance dose to be 1.86 mg (Belz 1968).

A more conservative approach achieves therapeutic levels by saturation-dosing over 4-5 days with 1.5 mg/d, followed by doses of 0.5-1.5 mg/d depending on individual tolerances. Because of the rapid excretion kinetics, slow ramping-up towards saturation doses (as it is usual practice with Digoxin) is not necessary. In cases of increased need for glycoside effect, daily doses of 2.0 or even 2.5 mg have been used in cardiac patients.

For clinical purposes in the cardiovascular field, the indirect determination of optimal circulating concentrations is more practical: the substance is injected intravenously at tolerable intervals up to a total dosage that produces the desired effect (in the case of Proscillaridin this could be for example the disappearance of atrial fibrillation); subsequently, the drug is given orally at sub-toxic doses until the same effect is achieved. This dose is the Effective oral Dose (ED_{p.o.}), which for Proscillaridin can go as high as 8.5 to 13.1 mg (total loading dose), depending on the speed of administration (2.25 mg/d for 4 days vs. 1.5 mg/d for 9 days), and from 0.65 up to 1.8 mg for maintenance of therapeutic levels (see for example: Gould L et al. 1971, or Bulitta A 1974).

For the present cancer indication, the effective oral dose is generally about 1.5-3 times for the cardiac indication. It is important to notice that, comparison studies between patients with cardiac insufficiency and cardiologically-normal individuals showed clearly that the latter have a much better tolerance for Proscillaridin before the onset of typical glycoside intoxication symptoms, changes in ECG, and metabolite profile (Gebhardt et al. 1965; Doneff et al. 1966); doses of up to 3.5 mg/d were well tolerated in cardiologically-normal individuals (Heierli et al. 1971).

However, in light of the often diminished body weight of cancer patients, and the fact that decreased stomach acid produces higher plasma concentrations, careful monitoring for appearance of toxic side effects at rapid saturation dosing will be essential in patients that fit these descriptions.

Toxicology:

The LD₅₀ p.o. in rats is reported as 0.25 mg/Kg in adult, and as 76 mg/Kg in young animals (female), making Proscillaridin about half as toxic as Digitoxin (0.1 mg/Kg/adult) (Goldenthal E I 1970). Rodents, however are bad toxicity indicators for cardiac glycosides because of their pronounced insensitivity towards this particular compound class (with the exception of Scillirosid, which is actually used as a rodenticide).

Intravenous toxicity in cats was determined to be 0.193 mg/Kg, positioning Proscillaridin in between Ouabain (0.133 mg/Kg) and Digoxin (0.307 mg/Kg). Duodenal administration, however, reverses this order, probably due to metabolic transformation during absorption by the gut wall. The values are: 5.3 mg/Kg for Ouabain, 1.05 mg/Kg for Proscillaridin, and 0.78 mg/Kg for Digoxin (Lenke D, Schneider B 1969/1970). Similar values were found in guinea pigs (Kurbjuweit H G 1964; Kobinger W. et al. 1970). These toxicology data helps to guide skilled artisans to set the upper limit dosage for the treatment of refractory cancers.

a) Acute Toxicity:

Proscillaridin exhibits about half the toxicity of Ouabain (Melville K I et al. 1966). The relatively wide therapeutic window of the compound in comparison to Ouabain or Digoxin is due to a combination of plasma-protein binding and rapid clearance (Kobinger W. et al. 1970); nevertheless, doses above 4 mg p.o./d in healthy individuals produce the for cardiac glycosides typical intoxication symptoms (nausea, headaches, seasickness, cardiac arrhythmias, bradycardia, extrasystoles).

However, the great advantage of Proscillaridin over other cardiac glycosides lies in the rapid clearance of the drug, so that toxic symptoms disappear very quickly after dosing is discontinued.

b) Chronic Toxicity:

Proscillaridin is still prescribed in Europe for the long-term medication of various cardiac illnesses. Patients take up to 1.5 mg per day without any negative side effects. The longest clinical and post-clinical observation of patients taking Proscillaridin was published in 1968:1067 patients were observed for up to 3 years after their initial dose, which was often a switch from Digitalis (Marx E. 1968). Of these only 0.7% developed negative side effects to such an extent that they had to be taken off the treatment. Upon reviewing the clinical safety data of Proscillaridin in a total of 3740 patients, Applicants found that none of these cases noted any long-term or late-appearing chronic toxicity.

c) Side Effects:

In healthy volunteers, 1.5 mg daily for 20 days produced no negative side effects (Andersson K E et al. 1975). Changes in color vision (Gebhardt et al. 1965) and other symptoms typical for Digitalis intoxication disappeared in patients after the switch from Digitalis to Proscillaridin. The only remarkable side effects that appear in almost all clinical reports at a level of 5% average are nausea, seasickness, headache, vomiting, stomach cramps and diarrhea (in order of decreasing frequency); very few patients develop cardiac arrhythmias or bradycardia. In most cases, these symptoms were of a transient nature, and could be controlled by temporarily lowering the administered dose. It must be mentioned, however, that in most instances the individuals under observation were very ill cardiac patients, which are known to have a higher sensitivity towards cardiac glycoside action and side-effects than cardiologically-healthy individuals.

In the clinical trial results study below, a small percentage (about 6.3%) of the patients also exhibited certain side-effects, the most negative symptoms being: nausea, stomach irritation, sea-sickness, diarrhea, cardiac arrhythmia, bradycardia, and extra-systoles. However, these symptoms are mostly transient. In >95% of the reported cases, therapy could be resumed after a brief hiatus.

d) Interactions with Other Drugs:

Possible negative interactions with other drugs are the same for Proscillaridin as with other cardiac glycosides such as Digoxin or Digitoxin. The corresponding precautions can be taken from the respective monographies in the Physician's Desk Reference. Coprescription of anti-hypertensives, vasodilators and diuretics are quite common with Proscillaridin. The molecular mechanism of action involves modulation of the Na/K-ATPase ion-pump (see above paragraph), resulting in a net loss of intracellular potassium and an increase of this ion in the plasma. Therefore, the possibility of hyperkalemia, especially during the loading phase of the treatment with Proscillaridin, warrants careful monitoring of electrolyte levels. Thus in certain embodiments, the method of the invention include a further step of monitoring electrolyte levels in patients subject to the treatment to avoid or ensure early detection of hyperkalemia and other associated side-effects.

On the other hand, when diuretics are being used concomitantly, the danger of alkalosis exists, and K and Cl must eventually be replaced. Quinidine, used as an anti-arrhythmic, diminishes hepatic excretion of Proscillaridin, and blood plasma levels might rise accordingly.

Cardiac glycosides, in conjunction with vasodilators and diuretics, have shown beneficial effects on myocardial failure scenarios in cancer patients after radiation or doxorubicin therapy (for example: Haq M M et al. 1985; Schwartz R G et al. 1987; Cordioli E et al. 1997).

Clinical Safety

Clinical safety of the subject cardiac glycosides, particularly safety in severely ill patient populations, including cancer patients, has also been evaluated.

Applicants have reviewed clinical trial results compiled from 47 clinical studies from the years 1964 to 1977. These studies describe a total of 3740 patients on Proscillaridin A treatment over an observation period of as long as 3 years. The studies were especially analyzed for the observation of acute or chronic negative side effects in relation to the initial diagnoses present at commencement of the medication.

Also noted are any concomitant medications to detect any incompatibilities. In most of the analyzed studies the patient population consisted of seriously ill individuals: besides severe heart conditions, many patients had concomitant diagnoses ranging from diabetes-mellitus, liver cirrhosis, hypertension, pulmonary and/or hepatic edema, bronchial emphysema, kidney failure, gastritis, stomach ulcers, and/or severe obesity.

Despite the general poor condition of these patients, and in respect to the present study, it is important to notice that the large majority of these severely ill patients tolerated Proscillaridin A very well. Proscillaridin A was well-tolerated at ˜1.5 mg/d in these cardiac patients, and up to about 3.5 mg/d in cardiologically normal individuals.

For example, in one of the studies reviewed (Bierwag K 1970), Proscillaridin was given to non-cardiac patients as a prophylactic to prevent occurrence of cardiac complications during and after impending surgery. The 50 patients described ranged in age from 50 to 83 years. The majority were cancer patients with the following diagnoses:
Gall bladder carcinoma
Papillary carcinoma
Stomach carcinoma
Colorectal adenocarcinoma
Mamma carcinoma

The patients received 0.25 to 0.5 mg/d intra-venously for four days before surgery and 0.25 mg/d during the four following days; they were then switched to an oral dose of 0.75 to 1.5 mg/d.

Considering the pharmacokinetic characteristics of Proscillaridin described above, 0.5 mg/d i.v./4d is equivalent to an oral dose for loading of roughly 2.5 mg/d for three days, or 1.8 mg/d for 4 days. This dose was well tolerated by all cancer patients with no appearance of either gastrointestinal or cardiac side effects.

### Example XIX

### Estimation of Therapeutic Index from Steady State Delivery of Compounds in Mice

To estimate the therapeutic index of the subject cardiac glycosides, we measured the therapeutic serum concentrations of the subject cardiac glycosides (e.g., BNC-1 and BNC-4) required to achieve greater than 60% tumor growth inhibition (TGI), and the corresponding toxic serum concentrations for these cardiac glycosides.

For BNC-1, the therapeutic serum concentration required to achieve >60% TGI is about 20±15 ng/ml, while the toxic serum concentration at day 1 is about 50±21 ng/ml. Therefore, the therapeutic index (toxic concentration/therapeutic level) for BNC-1 is about 2.5.

In contrast, for BNC-4, the therapeutic serum concentration required to achieve >60% TGI is about 48±23 ng/ml, while the toxic serum concentration at day 1 is about 518±121 ng/ml. Therefore, the therapeutic index (toxic concentration/therapeutic level) for BNC-4 is about 10.79. This suggests that BNC-4 and other bufadienolides and aglycones thereof generally have higher therapeutic index, and are preferred over the cardenolides.

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A method of inhibiting the growth or spread of a refractory cancer in an individual, comprising administering to the individual a Na⁺/K⁺-ATPase inhibitor in an oral dosage form over a treatment period, wherein the oral dosage form maintains an effective steady state serum concentration of from about 10 to about 700 ng/mL.

2-4. (canceled)

5. A method of inhibiting the growth or spread of a refractory cancer in an individual, comprising administering to the individual a Na⁺/K⁺-ATPase inhibitor in an oral dosage form over a treatment period, wherein the oral dosage form comprises a total daily dose of from about 2.25 to about 7.5 mg per human individual.

6-31. (canceled)

32. A method of inhibiting the growth or spread of a refractory cancer in an individual, comprising administering to the individual scillaren in an oral dosage form over a treatment period.

33. A method for promoting treatment of an individual suffering from a refractory cancer, comprising packaging, labeling and/or marketing a Na⁺/K⁺-ATPase inhibitor in an oral dosage form to be used as part of a treatment for inhibiting the growth or spread of the refractory cancer over a treatment period, wherein the oral dosage form maintains an effective steady state serum concentration of from about 10 to about 700 ng/mL.

34-36. (canceled)

37. A method for promoting treatment of an individual suffering from a refractory cancer, comprising packaging, labeling and/or marketing a Na⁺/K⁺-ATPase inhibitor in an oral dosage form to be used as part of a treatment for inhibiting the growth or spread of the refractory cancer over a treatment period, wherein the oral dosage form comprises a total daily dose of from about 2.25 to about 7.5 mg per human individual.

38-63. (canceled)

64. A method for promoting treatment of an individual suffering from a refractory cancer, comprising packaging, labeling and/or marketing scillaren in an oral dosage form to be used as part of a treatment for inhibiting the growth or spread of the refractory cancer over a treatment period.

65. A method of treating multidrug resistance of refractory tumor cells in a refractory cancer patient in need of such treatment, said method comprising administering, concurrently or sequentially, an effective amount of a Na⁺/K⁺-ATPase inhibitor in an oral dosage form and an antineoplastic agent to said patient over a treatment period, wherein the oral dosage form maintains an effective steady state serum concentration of from about 10 to about 700 ng/mL.

66-68. (canceled)

69. A method of treating multidrug resistance of refractory tumor cells in a refractory cancer patient in need of such treatment, said method comprising administering, concurrently or sequentially, an effective amount of a Na⁺/K⁺-ATPase inhibitor in an oral dosage form and an antineoplastic agent to said patient over a treatment period, wherein the oral dosage form comprises a total daily dose of from about 2.25 to about 7.5 mg per human individual.

70-95. (canceled)

96. A method of treating multidrug resistance of refractory tumor cells in a refractory cancer patient in need of such treatment, said method comprising administering, concurrently or sequentially, an effective amount of scillaren in an oral dosage form and an antineoplastic agent to said patient over a treatment period.

97. A packaged pharmaceutical comprising a Na⁺/K⁺-ATPase inhibitor formulated as an oral dosage form in a pharmaceutically acceptable excipient and suitable for use in humans, and a label or instructions for administering the Na⁺/K⁺-ATPase inhibitor as part of a treatment for inhibiting the growth or spread of a refractory cancer over a treatment period, wherein the oral dosage form maintains an effective steady state serum concentration of from about 10 to about 700 ng/mL.

98-100. (canceled)

101. A packaged pharmaceutical comprising a Na⁺/K⁺-ATPase inhibitor formulated as an oral dosage form in a pharmaceutically acceptable excipient and suitable for use in humans, and a label or instructions for administering the Na⁺/K⁺-ATPase inhibitor as part of a treatment for inhibiting the growth or spread of a refractory cancer over a treatment period, wherein the oral dosage form comprises a total daily dose of from about 2.25 to about 7.5 mg per human individual.

102-127. (canceled)

128. A packaged pharmaceutical comprising scillaren formulated as an oral dosage form in a pharmaceutically acceptable excipient and suitable for use in humans, and a label or instructions for administering the scillaren as part of a treatment for inhibiting the growth or spread of a refractory cancer over a treatment period.

129. Use of a Na⁺/K⁺-ATPase inhibitor in the manufacture of a medicament in oral dosage form, for treating or inhibiting the growth or spread of a refractory cancer in an individual over a treatment period, wherein the oral dosage form maintains an effective steady state serum concentration of from about 10 to about 700 ng/mL.

130. (canceled)

131. Use of a Na⁺/K⁺-ATPase inhibitor in the manufacture of a medicament in oral dosage form, for treating/inhibiting the growth or spread of a refractory cancer in an individual over a treatment period, wherein the oral dosage form comprises a total daily dose of from about 2.25 to about 7.5 mg per human individual.

132. (canceled)

133. Use of scillaren in the manufacture of a medicament in oral dosage form, for treating/inhibiting the growth or spread of a refractory cancer in an individual over a treatment period.

134. Use of a Na⁺/K⁺-ATPase inhibitor in the packaging, labeling and/or marketing of an oral dosage form medicament, for treating/inhibiting the growth or spread of a refractory cancer in an individual over a treatment period, wherein the oral dosage form maintains an effective steady state serum concentration of from about 10 to about 700 ng/mL.

135. Use of a Na⁺/K⁺-ATPase inhibitor in the packaging, labeling and/or marketing of an oral dosage form medicament, for treating/inhibiting the growth or spread of a refractory cancer in an individual over a treatment period, wherein the oral dosage form comprises a total daily dose of from about 2.25 to about 7.5 mg per human individual.

136. Use of scillaren in the packaging, labeling and/or marketing of an oral dosage form medicament, for treating/inhibiting the growth or spread of a refractory cancer in an individual over a treatment period.

137. Use of a Na⁺/K⁺-ATPase inhibitor in the manufacture of a medicament in oral dosage form, for treating multidrug resistance of refractory tumor cells in a refractory cancer patient in need of such treatment, the Na⁺/K⁺-ATPase inhibitor being administered, concurrently or sequentially, with an anti-neoplastic agent to the patient, wherein the oral dosage form maintains an effective steady state serum concentration of from about 10 to about 700 ng/mL.

138. (canceled)

139. Use of a Na⁺/K⁺-ATPase inhibitor in the manufacture of a medicament in oral dosage form, for treating multidrug resistance of refractory tumor cells in a refractory cancer patient in need of such treatment, the Na⁺/K⁺-ATPase inhibitor being administered, concurrently or sequentially, with an anti-neoplastic agent to the patient, wherein the oral dosage form comprises a total daily dose of from about 2.25 to about 7.5 mg per human individual.

140. (canceled)

141. Use of scillaren in the manufacture of a medicament in oral dosage form, for treating multidrug resistance of refractory tumor cells in a refractory cancer patient in need of such treatment, the Na⁺/K⁺-ATPase inhibitor being administered, concurrently or sequentially, with an anti-neoplastic agent to the patient.

142. A method of treating multidrug resistance of refractory tumor cells in a refractory cancer patient in need of such treatment, said method comprising administering, concurrently or sequentially, an effective amount of a Na⁺/K⁺-ATPase inhibitor in an oral dosage form and an antineoplastic agent to said patient over a treatment period, said refractory cancer is lung or renal cancer.

143. (canceled)

144. Use of a Na⁺/K⁺-ATPase inhibitor in the manufacture of a medicament in an oral dosage form, for treating multidrug resistance of refractory tumor cells in a refractory lung or renal cancer patient in need of such treatment, said medicament is administered, either concurrently or sequentially, with an antineoplastic agent over a treatment period.

145. A pharmaceutical composition comprising a bufadienolide Na⁺/K⁺-ATPase inhibitor or aglycone thereof, formulated in a pharmaceutically acceptable excipient and suitable for use in humans, wherein said bufadienolide or aglycone thereof is a solid oral dosage form of at least about 1.5 mg.

146-148. (canceled)
